SEQUENCE LISTING (48 sequences)

SEQ ID NO: 1 - DNA, 1059 bp, Pseudomonas putida
  source 1..1059: mol_type = genomic DNA
  CDS 1..1059
atgagcagttcaccaacgatttccccggccagcgatacgttcgcggccatgactgacgatcaccgcctggccgagttcatccgcgagcaggcctcggcaacgcgggtggtcatccaggcgcgcaagcgcctgagcggcggcgctatccaggaaaactggctgctggacctgctgatcgaaggcggcccgtgggccggtgtccggcgttgggtactgcgcagcgatgcgctttcagcgctacccgccagccttgaccgtgaacaggagttcgccgtgctgcaggtggtttaccaggccggcgtgaaagtgccacgcccgctctggctgtgccgcgatgtgcgcgtgcatgggcgggtgttcttcctgatggagtatgtgccgggtagcgctgccggccgcgcgctcagcaccggcgccggtcctcagggccgggcgcaactggcgacgcagcttggcgccaacctggcgcgtctgcatcaggtccgcccgccgtgcgccacgctgtgcttcctgtccgttccggacagctcgccggccctggcgaccatcgacgcctaccgccgctacctcgacaccctcgccgatgcctatccggtgctggaatggggcctgcgctggtgcgagctgcatgcgccgcgcagcagcaccctgtgcctgttgcaccgtgactaccgcaccggcaactacctggccagcgaagaagggctggaggccgtgctcgactgggagttcaccggctggggagatccttgcgaggacctcggctggttcaccgcccgttgctggcgttttacccgtccagacctcgaagccggcggcattggccagctggaggattttctgcgtggttatcacgaggtgtcttcgctgtgcatcgagcgcagtcggctccactactggcaagtcatggccaccctgcgctgggcggtgattgccttgcagcaagggcagcgccatctgtccggtgaagaaccgtcgctcgagctagcactgacagcccggctgttgccggagctcgaactcgacatcctgcacatgaccggagccgaagcgccatga

SEQ ID NO: 2 - protein, 352 aa, Pseudomonas putida
MSSSPTISPASDTFAAMTDDHRLAEFIREQASATRVVIQARKRLSGGAIQENWLLDLLIEGGPWAGVRRWVLRSDALSALPASLDREQEFAVLQVVYQAGVKVPRPLWLCRDVRVHGRVFFLMEYVPGSAAGRALSTGAGPQGRAQLATQLGANLARLHQVRPPCATLCFLSVPDSSPALATIDAYRRYLDTLADAYPVLEWGLRWCELHAPRSSTLCLLHRDYRTGNYLASEEGLEAVLDWEFTGWGDPCEDLGWFTARCWRFTRPDLEAGGIGQLEDFLRGYHEVSSLCIERSRLHYWQVMATLRWAVIALQQGQRHLSGEEPSLELALTARLLPELELDILHMTGAEAP

SEQ ID NO: 3 - DNA, 357 bp, Pseudomonas putida
  source 1..357: mol_type = genomic DNA
  CDS 1..357
atgacccaacccaacgcccacgaattgctcgagatcgcccgcgcgacgcttctggagcagctgctgccagcgctgcccggcgagttgcgttacccggccctgatgatcgccaacgccatggccattgcggcccgcgaaaaccgcttgggcgctcaggccgaggatcaggagcaggcgcgtctggccgccttggtcgatgacgcgccgtcgacattgcccgacctgcgccgccaactggctcgcgccattcgccagggcagccatgacgccccgcaaacccggcgcaccctggtcgagacattacgccagatcaccgttgcccgattggcgatcagcaaccccaaggccttgccctga

SEQ ID NO: 4 - protein, 118 aa, Pseudomonas putida
MTQPNAHELLEIARATLLEQLLPALPGELRYPALMIANAMAIAARENRLGAQAEDQEQARLAALVDDAPSTLPDLRRQLARAIRQGSHDAPQTRRTLVETLRQITVARLAISNPKALP

SEQ ID NO: 5 - DNA, 1173 bp, Pseudomonas putida
  source 1..1173: mol_type = genomic DNA
  CDS 1..1173
atgaacttcactctcccggacgaactgctcgccttgcaggccaagactcgagacttcattgccgaacaggtcatcccattcgagaacgacccccgccagaacagccacggccccagcgacgcactgcgccaggacctggtgctctgcgcccgcgccgctggcttgctgacgcctcacgccagccgcgaaatgggcggtctggaactgagccatgtggccaaggcgatcgtcttcgaagaagccggctactcgccgctgggcccggtagcgctgaatatccatgcgccggacgaaggcaatatccacctgatggacgtggtcgccaccgaagcgcagaaggaccgctggttgcgcccgctggtccagggccatgcccgttcgtgcttcgccatgacggagcctgctccgggctccggttcggatccgtcgatgctgcgcaccactgccacccgcgatggcgacgactacctgatcaatggtcgcaagtggctgatcaccggggccgaaggcgcggacttcggcatcatcatggcgcgcatggaggacggcaccgcgaccatgttcctgaccgacatgaagcgcgacggcatcatccatgaacgtcagctggactcgctggacagctgttttaccggcggtcacgggcagctgcgtttcgacaacctgcgtattccggcgagcgatgtcctcggcgagatcggcaagggcttccggtatgcccaggtgcgcctggcgcctgcacgcttgactcattgcatgcgctggctcggtgccgcgcgccgcgcccacgacatcgcctgcgactatgcgcgcacccgggacgcctttggcaagccgctgggcgagcaccagggcgtgggtttcatgctggccgacaacatgatggacctgcacgtggtgcgtctggcggtctggcactgcgcctgggtgctcgaccagggccggcgcgccaatgtcgattcgagcatggccaaggtgatcagcgccgaggcgctgtggcgggtggtcgatcgttgcgtccaggtattgggtggacgcggggtgaccggggacaccgtggtggagcggatcttccgcgacattcgcccgttccgcatctatgacggcccgagcgaagtgcaccgcatgagcctggcgaagaagctgctcgaccagcgcctggaggcccactga

SEQ ID NO: 6 - protein, 390 aa, Pseudomonas putida
MNFTLPDELLALQAKTRDFIAEQVIPFENDPRQNSHGPSDALRQDLVLCARAAGLLTPHASREMGGLELSHVAKAIVFEEAGYSPLGPVALNIHAPDEGNIHLMDVVATEAQKDRWLRPLVQGHARSCFAMTEPAPGSGSDPSMLRTTATRDGDDYLINGRKWLITGAEGADFGIIMARMEDGTATMFLTDMKRDGIIHERQLDSLDSCFTGGHGQLRFDNLRIPASDVLGEIGKGFRYAQVRLAPARLTHCMRWLGAARRAHDIACDYARTRDAFGKPLGEHQGVGFMLADNMMDLHVVRLAVWHCAWVLDQGRRANVDSSMAKVISAEALWRVVDRCVQVLGGRGVTGDTVVERIFRDIRPFRIYDGPSEVHRMSLAKKLLDQRLEAH

SEQ ID NO: 7 - DNA, 768 bp, Pseudomonas putida
  source 1..768: mol_type = genomic DNA
  CDS 1..768
atgcagccgaaccttgcccgactgttcgccctcgacgggcgtcgcgccctggtgaccggggcctccagcggcctgggccgtcacttcgccatgaccctggccgccgcaggcgccgaggtggtggtgaccgccagacgccaggcgccgctgcaggcgttggtggaggccatcgaggtggccggagggcgggcgcaggcctttgccctcgatgtgacgagccgtgaggacatctgccgggtgctcgatgccgccggcccgctggatgttctggtcaacaatgcgggggtgagcgacagccagcctttgctagcctgcgatgatcaaacctgggaccacgtgctcgacaccaacctcaagggcgcctgggccgtggcccaggaaagcgcccggcgcatggtggtggcggggaaggggggcagcctgatcaatgtcacctcgatcctcgccagccgtgtggccggcgccgtcggcccttacctggcggccaaggccggcctggcccacctgacccgcgccatggcgctggagttggcgcgccatggtatccgggtgaacgccctggcgcccggctacgtgatgactgatttgaacgaggccttcctggccagcgaggccggtgacaagttgcgctcgcggatccccagccgccgcttcagcgtgccgtcggacctggacggcgccttgctgctgctcgccagcgatgccgggcgggcgatgagcggcgctgagatcgtggtcgatggcggccacctgtgcagcagcctgtaa

SEQ ID NO: 8 - protein, 255 aa, Pseudomonas putida
MQPNLARLFALDGRRALVTGASSGLGRHFAMTLAAAGAEVVVTARRQAPLQALVEAIEVAGGRAQAFALDVTSREDICRVLDAAGPLDVLVNNAGVSDSQPLLACDDQTWDHVLDTNLKGAWAVAQESARRMVVAGKGGSLINVTSILASRVAGAVGPYLAAKAGLAHLTRAMALELARHGIRVNALAPGYVMTDLNEAFLASEAGDKLRSRIPSRRFSVPSDLDGALLLLASDAGRAMSGAEIVVDGGHLCSSL

SEQ ID NO: 9 - DNA, 1647 bp, Pseudomonas putida
  source 1..1647: mol_type = genomic DNA
  CDS 1..1647
atgatggttccaaccctcgaacacgagcttgctcccaacgaagccaaccatgtcccgctgtcgccgctgtcgttcctcaagcgtgccgcgcaggtgtacccgcagcgcgatgcggtgatctatggcgcaaggcgctacagctaccgtcagttgcacgagcgcagccgcgccctggccagtgccttggagcgggtcggtgttcagccgggcgagcgggtggcgatattggcgccgaacatcccggaaatgctcgaggcccactatggcgtgcccggtgccggggcggtgctggtgtgcatcaacatccgcctggaggggcgcagcattgccttcatcctgcgtcactgcgcggccaaggtattgatctgcgatcgtgagttcggtgccgtggccaatcaggcgctggccatgctcgatgcgccgcccttgctggtgggcatcgacgatgatcaggccgagcgcgccgatttggcccacgacctggactacgaagcgttcttggcccagggcgaccccgcgcggccgttgagtgcgccacagaacgaatggcagtcgatcgccatcaactacacctccggcaccacgggggaccccaagggcgtggtgctgcatcaccgcggcgcctacctcaacgcctgcgccggggcgctgatcttccagttggggccgcgcagcgtctacttgtggaccttgccgatgttccactgcaacggctggagccatacctgggcggtgacgttgtccggtggcacccacgtgtgtctgcgcaaggtccagcctgatgcgatcaacgccgccatcgccgagcatgccgtgactcacctgagcgccgccccagtggtgatgtcgatgctgatccacgccgagcatgccagcgcccctccggtgccggtttcggtgatcactggcggtgccgccccgcccagtgcggtcatcgcggcgatggaggcgcgtggcttcaacatcacccatgcctatggcatgaccgaaagctacggtcccagcacattgtgcctgtggcagccgggtgtcgacgagttgccgctggaggcccgggcccagttcatgagccgccagggcgtcgcccacccgctgctcgaggaggccacggtgctggataccgacaccggccgcccggtcccggccgacggccttaccctcggcgagctggtggtgcggggcaacactgtgatgaaaggctacctgcacaacccagaggctacccgtgccgcgttggccaacggctggctgcacacgggcgacctggccgtgctgcacctggacggctatgtggaaatcaaggaccgagccaaggacatcatcatttctggcggcgagaacatcagttcgctggagatagaagaagtgctctaccagcaccccgaggtggtcgaggctgcggtggtggcgcgtccggattcgcgctggggcgagacacctcacgctttcgtcacgctgcgcgctgatgcactggccagcggggacgacctggtccgctggtgccgtgagcgtctggcgcacttcaaggcgccgcgccatgtgtcgctcgtggacctgcccaagaccgccactggaaaaatacagaagttcgtcctgcgtgagtgggcccggcaacaggaggcgcagatcgccgacgccgagcattga

SEQ ID NO: 10 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 11 - DNA, 20 bp, synthetic construct
  misc_feature 1..20: note = PCR Primer
  source 1..20: mol_type = other DNA
aacctggacggtgaagagcg

SEQ ID NO: 12 - DNA, 19 bp, synthetic construct
  misc_feature 1..19: note = PCR Primer
  source 1..19: mol_type = other DNA
gaacggacaggaagcacag

SEQ ID NO: 13 - DNA, 32 bp, synthetic construct
  misc_feature 1..32: note = PCR Primer
  source 1..32: mol_type = other DNA
ggccacgcgtcgactagtaccccccccccccc

SEQ ID NO: 14 - DNA, 36 bp, synthetic construct
  misc_feature 1..36: note = PCR Primer
  source 1..36: mol_type = other DNA
ggccacgcgtcgactagtacggghhggghhggghhg

SEQ ID NO: 15 - DNA, 21 bp, synthetic construct
  misc_feature 1..21: note = PCR Primer
  source 1..21: mol_type = other DNA
ccaatgcccgtagcaggtcgc

SEQ ID NO: 16 - DNA, 21 bp, synthetic construct
  misc_feature 1..21: note = PCR Primer
  source 1..21: mol_type = other DNA
gaactcctgttcacggtcaag

SEQ ID NO: 17 - DNA, 19 bp, synthetic construct
  misc_feature 1..19: note = PCR Primer
  source 1..19: mol_type = other DNA
tcaatgatcgacggcaccg

SEQ ID NO: 18 - DNA, 21 bp, synthetic construct
  misc_feature 1..21: note = PCR Primer
  source 1..21: mol_type = other DNA
acgctgtgcttcctgtccgtt

SEQ ID NO: 19 - DNA, 20 bp, synthetic construct
  misc_feature 1..20: note = PCR Primer
  source 1..20: mol_type = other DNA
gttcttcaccggacagatgg

SEQ ID NO: 20 - DNA, 20 bp, synthetic construct
  misc_feature 1..20: note = PCR Primer
  source 1..20: mol_type = other DNA
cccacgaattgctcgagatc

SEQ ID NO: 21 - DNA, 18 bp, synthetic construct
  misc_feature 1..18: note = PCR Primer
  source 1..18: mol_type = other DNA
gcaggtcgggcaatgtcg

SEQ ID NO: 22 - DNA, 18 bp, synthetic construct
  misc_feature 1..18: note = PCR Primer
  source 1..18: mol_type = other DNA
catgcccgttcgtgcttc

SEQ ID NO: 23 - DNA, 21 bp, synthetic construct
  misc_feature 1..21: note = PCR Primer
  source 1..21: mol_type = other DNA
caggtccatcatgttgtcggc

SEQ ID NO: 24 - DNA, 19 bp, synthetic construct
  misc_feature 1..19: note = PCR Primer
  source 1..19: mol_type = other DNA
acgagccgtgaggacatct

SEQ ID NO: 25 - DNA, 18 bp, synthetic construct
  misc_feature 1..18: note = PCR Primer
  source 1..18: mol_type = other DNA
cgagcgcaacttgtcacc

SEQ ID NO: 26 - DNA, 20 bp, synthetic construct
  misc_feature 1..20: note = PCR Primer
  source 1..20: mol_type = other DNA
gctggtgtgcatcaacatcc

SEQ ID NO: 27 - DNA, 20 bp, synthetic construct
  misc_feature 1..20: note = PCR Primer
  source 1..20: mol_type = other DNA
gcagtggaacatcggcaagg

SEQ ID NO: 28 - DNA, 19 bp, synthetic construct
  misc_feature 1..19: note = PCR Primer
  source 1..19: mol_type = other DNA
tgttatacgcgcgtgttcg

SEQ ID NO: 29 - DNA, 20 bp, synthetic construct
  misc_feature 1..20: note = PCR Primer
  source 1..20: mol_type = other DNA
ggtacacgtagaacgccgac

SEQ ID NO: 30 - DNA, 22 bp, synthetic construct
  misc_feature 1..22: note = PCR Primer
  source 1..22: mol_type = other DNA
catggtgttcgtgctgttcacc

SEQ ID NO: 31 - DNA, 20 bp, synthetic construct
  misc_feature 1..20: note = PCR Primer
  source 1..20: mol_type = other DNA
gccgaacagcaacctgatca

SEQ ID NO: 32 - DNA, 21 bp, synthetic construct
  misc_feature 1..21: note = PCR Primer
  source 1..21: mol_type = other DNA
acgctgtgcttcctgtccgtt

SEQ ID NO: 33 - DNA, 17 bp, synthetic construct
  misc_feature 1..17: note = PCR Primer
  source 1..17: mol_type = other DNA
caggtcgggcaatgtcg

SEQ ID NO: 34 - DNA, 20 bp, synthetic construct
  misc_feature 1..20: note = PCR Primer
  source 1..20: mol_type = other DNA
cccacgaattgctcgagatc

SEQ ID NO: 35 - DNA, 19 bp, synthetic construct
  misc_feature 1..19: note = PCR Primer
  source 1..19: mol_type = other DNA
gaagcacgaacgggcatgg

SEQ ID NO: 36 - DNA, 21 bp, synthetic construct
  misc_feature 1..21: note = PCR Primer
  source 1..21: mol_type = other DNA
gccgacaacatgatggacctg

SEQ ID NO: 37 - DNA, 22 bp, synthetic construct
  misc_feature 1..22: note = PCR Primer
  source 1..22: mol_type = other DNA
cgtggtcccaggtttgatcatc

SEQ ID NO: 38 - DNA, 20 bp, synthetic construct
  misc_feature 1..20: note = PCR Primer
  source 1..20: mol_type = other DNA
gctcgacaccaacctcaagg

SEQ ID NO: 39 - DNA, 20 bp, synthetic construct
  misc_feature 1..20: note = PCR Primer
  source 1..20: mol_type = other DNA
gccaagaacgcttcgtagtc

SEQ ID NO: 40 - DNA, 20 bp, synthetic construct
  misc_feature 1..20: note = PCR Primer
  source 1..20: mol_type = other DNA
cacggtgctggataccgaca

SEQ ID NO: 41 - DNA, 20 bp, synthetic construct
  misc_feature 1..20: note = PCR Primer
  source 1..20: mol_type = other DNA
ggtacacgtagaacgccgac

SEQ ID NO: 42 - DNA, 19 bp, synthetic construct
  misc_feature 1..19: note = PCR Primer
  source 1..19: mol_type = other DNA
tgttatacgcgcgtgttcg

SEQ ID NO: 43 - DNA, 20 bp, synthetic construct
  misc_feature 1..20: note = PCR Primer
  source 1..20: mol_type = other DNA
gccgaacagcaacctgatca

SEQ ID NO: 44 - DNA, 81 bp, synthetic construct
  misc_feature 1..81: note = BarSeq Oligo
  misc_difference 59..63: note = n is a, c, g, or t
  source 1..81: mol_type = other DNA
aatgatacggcgaccaccgagatctacactctttccctacacgacgctcttccgatctnnnnngtcgacctgcagcgtacg

SEQ ID NO: 45 - DNA, 80 bp, synthetic construct
  misc_feature 1..80: note = BarSeq Oligo
  misc_difference 59..62: note = n is a, c, g, or t
  source 1..80: mol_type = other DNA
aatgatacggcgaccaccgagatctacactctttccctacacgacgctcttccgatctnnnngtcgacctgcagcgtacg

SEQ ID NO: 46 - DNA, 79 bp, synthetic construct
  misc_feature 1..79: note = BarSeq Oligo
  misc_difference 59..61: note = n is a, c, g, or t
  source 1..79: mol_type = other DNA
aatgatacggcgaccaccgagatctacactctttccctacacgacgctcttccgatctnnngtcgacctgcagcgtacg

SEQ ID NO: 47 - DNA, 78 bp, synthetic construct
  misc_feature 1..78: note = BarSeq Oligo
  misc_difference 59..60: note = n is a, c, g, or t
  source 1..78: mol_type = other DNA
aatgatacggcgaccaccgagatctacactctttccctacacgacgctcttccgatctnngtcgacctgcagcgtacg

SEQ ID NO: 48 - DNA, 242 bp, Pseudomonas putida
  source 1..242: mol_type = genomic DNA
ggcaagagcatccatcacggggcctgttcatgtttgaaatgtttcgataatgaatcaatagcttgcaaatatgcaacaagcagcctgactttcgctcggcggtgaggcgtcagaacccaggctgtatgcggcctgtggctgacttagagttggcccaggccttgctcttgggcttgtgccaacccagaaaccaaaggcagggcctacagaaccatgagcagttcaccaacgatttccccggc

(19) United States
(12) Patent Application Publication
(10) Pub. No.: US 2025/0163478 A1
(43) Pub. Date: May 22, 2025

(54) BIOCONVERSION OF LEVULINIC ACID IN GENETICALLY ENGINEERED HOSTS

(71) Applicant: Wisconsin Alumni Research Foundation, Madison, WI (US)

(72) Inventors: Brian Frederick Pfleger, Madison, WI (US); Jacqueline Marie Rand, Madison, WI (US); Christopher Robert Mehrer, Madison, WI (US); Matthew Ryan Incha, Berkeley, CA (US)

(73) Assignee: Wisconsin Alumni Research Foundation, Madison, WI (US)

(21) Appl. No.: 18/884,266

(22) Filed: Sep. 13, 2024

Related U.S. Application Data
(62) Division of application No. 17/159,850, filed on Jan. 27, 2021, now Pat. No. 12,116,609.
Application No. 17/159,850 is a division of application No. 16/135,123, filed on Sep. 19, 2018, now Pat. No. 10,934,563.
(60) Provisional application No. 62/560,247, filed on Sep. 19, 2017.

(51) Int. Cl. C12P 7/16; C12N 9/10; C12N 9/18; C12N 15/52; C12N 15/62; C12N 15/63; C12N 15/64; C12N 15/66; C12N 15/69; C12P 7/26

(52) U.S. Cl. CPC C12P 7/16; C12N 9/1029; C12N 9/18; C12N 15/52; C12N 15/62; C12N 15/63; C12N 15/64; C12N 15/66; C12N 15/69; C12P 7/26; C12Y 203/01009; C12Y 208/03; C12Y 301/01

(57) ABSTRACT

Described is a recombinant expression vector that enables a cell transformed to contain and express the vector to use levulinic acid as a carbon source, thereby converting levulnic acid into 2-butanne. Also described are genetically modified cells transformed to contain and express the vector and methods of using the cells to produce 2-butanone from a medium containing levulinic acid.

### CROSS-REFERENCE TO RELATED APPLICATIONS

This is a divisional of co-pending application Ser. No. 17/159,850, filed Jan. 27, 2021, which is a divisional of co-pending application Ser. No. 16/135,123, filed Sep. 19, 2018, now U.S. Pat. No. 10,934,563, issued Mar. 2, 2021, which claims priority to provisional application Ser. No. 62/560,247, filed Sep. 19, 2017, which are incorporated herein by reference.

### FEDERAL FUNDING STATEMENT

This invention was made with government support under CBET1149678 awarded by the National Science Foundation. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Jan. 29, 2025, is named “REPLACEMENT_SEQ_LIST—09824566-P170368US04.xml” and is 66,396 bytes in size.

### BACKGROUND

Levulinic acid (LA) is a five carbon γ-keto acid that can be readily obtained from biomass through non-enzymatic, acid hydrolysis of a wide range of feedstocks. LA was named one of the US Department of Energy's “Top 12 value-added chemicals from biomass” because it can be used as a renewable feedstock for generating a variety of molecules, such as fuel additives, flavors, fragrances and polymers, through chemical catalysis. In addition, microbes can use LA as a sole carbon source and have been shown to convert LA into polyhydroxyalkanoates, short chain organic acids, and trehalose. (Chung, S. H., Choi, G. G., Kim, H. W. & Rhee, Y. H. Effect of Levulinic Acid on the Production of Poly (3-hydroxybutyrate-co-3-hydroxyvalerate) by Ralstonia eutropha KHB-8862. Society 39, 79-82 (2001). Berezina, N. & Yada, B. Improvement of the poly (3-hydroxybutyrate-co-3-hydroxyvalerate) (PHBV) production by dual feeding with levulinic acid and sodium propionate in Cupriavidus necator. N. Biotechnol. 33, 231-236 (2016). Valentin, H. E., Schönebaum, A. & Steinbüchel, A. Identification of 5-hydroxyhexanoic acid, 4-hydroxyheptanoic acid and 4-hydroxyoctanoic acid as new constituents of bacterial polyhydroxyalkanoic acids. Appl. Microbiol. Biotechnol. 46, 261-267 (1996). Jang, J. H. & Rogers, P. L. Effect of levulinic acid on cell growth and poly-beta-hydroxyalkanoate production by Alcaligenes sp SH-69. J. Chem. Inf. Model. 18, 219-224 (1996). Habe, H. et al. Bacterial production of short-chain organic acids and trehalose from levulinic acid: A potential cellulose-derived building block as a feedstock for microbial production. Bioresour. Technol. 177, 381-386 (2015). Martin, C. H., Wu, D., Prather, K. L. J. & Jones Prather, K. L. Integrated bioprocessing for the pH-dependent production of 4-valerolactone from levulinate in pseudomonas putida KT2440. Appl. Environ. Microbiol. 76, 417-424 (2010). Yeon, Y. J., Park, H. Y. & Yoo, Y. J. Enzymatic reduction of levulinic acid by engineering the substrate specificity of 3-hydroxybutyrate dehydrogenase. Bioresour. Technol. 134, 377-380 (2013).) All of these bioconversion studies were conducted with natural bacterial isolates because the enzymes comprising a LA assimilation pathway were unknown. See Habe et al., supra. This knowledge gap limits metabolic engineering and the potential of creating novel LA bioconversions.

While the enzymes responsible for LA assimilation were unknown at the time of these bioconversion demonstrations, other studies identified putative intermediates and suggested pathways for LA catabolism. In a study where crude cell lysates of Cupriavidus necator were fed LA, the concentration of LA and free CoA decreased over time while acetyl-CoA and propionyl-CoA concentrations increased, suggesting that LA is catabolized via CoA thioesters like other short-chain organic acids. Jaremko, M. & Yu, J. The initial metabolic conversion of levulinic acid in Cupriavidus necator. J. Biotechnol. 155, 293-298 (2011). In a second study, cultures of Pseudomonas putida KT2440 expressing a heterologous TesB thioesterase were fed LA. Here, 4-hydroxyvalerate (4HV) and 3-hydroxyvalerate (3HV) transiently accumulated extracellularly before ultimately disappearing. Martin, C. H. & Prather, K. L. J. High-titer production of monomeric hydroxyvalerates from levulinic acid in Pseudomonas putida. J. Biotechnol. 139, 61-67 (2009). This observation strongly suggested that 4HV and 3HV (or their CoA thioesters) were pathway intermediates. Lastly, a metabolomic study of rat livers suggested that LA is catabolized to acetyl-CoA and propionyl-CoA via a unique phosphorylated acyl-CoA. (Zhang, G. F. et al. Catabolism of 4-hydroxyacids and 4-hydroxynonenal via 4-hydroxy-4-phosphoacyl-CoAs. J. Biol. Chem. 284, 33521-33534 (2009) and Harris, S. R. et al. Metabolism of levulinate in perfused rat livers and live rats: Conversion to the drug of abuse 4-hydroxypentanoate. J. Biol. Chem. 286, 5895-5904 (2011).) In sum, these observations suggest a relatively direct route from LA to beta-oxidation intermediates, but the enzymes comprising such a pathway remain unknown.

### SUMMARY

To utilize LA as a substrate for microbial growth or bioconversion, a detailed understanding of the metabolic pathway and enzymes involved is necessary. As disclosed herein, the genetic and biochemical factors that allow P. putida KT2440 to catabolize LA were elucidated. Using a loss of function screen of a transposon library, a putative LA utilization operon was identified, isolated, incorporated into an unnatural expression vector. The expression vector was used to transform heterologous hosts which the expressed the genes necessary for the transformed hosts to utilize LA as a carbon source. The operon consists of seven genes: two homologs for membrane transporters and five enzymatic proteins. The pathway was reconstituted in vitro. It was determined that all five enzymatic proteins are required for complete conversion of LA into 3HV-CoA, an intermediate in the β-oxidation of odd-chain fatty acids. A closer inspection of the CoA ligase encoded in the operon revealed a broad substrate promiscuity including C₄ to C₆ organic acids. A putative regulator proximal to the operon activated transcription of the LA catabolic genes in the presence of LA or 4HV. The induction tests revealed that while the CoA ligase might have nonspecific activity towards similar chain length acids, the promoter is only responsive when cells were provided LA or 4HV. The catabolism of LA to acetyl-CoA and propionyl-CoA requires at least 2 ATP that likely come from respiration and the tricarboxylic acid cycle.

Thus, a first version of the invention is a recombinant expression vector comprising at least one promoter operably linked to at least three of, at least four of, or all five of lvaA, lvaB, lvaC, lvaD, and lvaE. The promoter may be inducible or constitutively active. The expression vector may optionally comprise a nucleotide sequence encoding an acetoacetyl-CoA transferase, a short-chain thioesterase and/or a succinyl-CoA transferase, operably linked to a promoter. The recombinant expression vector might also optionally comprise a nucleotide sequence encoding an acetoacetate decarboxylase operably linked to a promoter. The recombinant expression may optionally further comprise a nucleotide sequence encoding FadB and/or FadJ, operably linked to a promoter.

Also disclosed herein is a genetically modified host cell transformed to contain and express a heterologous recombinant expression vector as described herein. The genetically modified host cell may optionally be fadE negative and/or atoC negative. Optionally, the genetically modified host cell may also be fadA, fadI or atoB negative. Optionally, the genetically modified host cell may also be fadR negative. In another version of the modified host, the host cell may optionally comprise an increased copy number of nucleotide sequences encoding FadB and/or FadJ as compared to the wild-type of the host cell.

The host cell may be selected from the group consisting of an archaeal cell, a bacterial cell, and a eukaryotic cell. Bacteria and eukaryotic single-cell organisms are preferred host cells. In some instances, the host cell may endogenously encode activities catalyzed by LvaAB, LvaC, LvaD, and/or LvaE in its genome. In these instances, the invention may rely on natively encoded activities rather than heterologous activities conferred by the vector expressing lvaA, lvaB, lvaC, lvaD, and lvaE.

The genetically modified host cell may optionally constitutively expresses acetoacetyl-CoA transferase.

Also disclosed herein is a method of catabolizing levulinic acid. That is, a method of enabling a host cell to use levulinic acid as a carbon source. The method comprises culturing a genetically modified host cell as disclosed herein in a medium containing levulinic acid, under conditions and for a time wherein at least a portion of the levulinic acid is catabolized by action of the genetically modified host cell.

Yet another method disclosed herein is a method of making 2-butanone. The method comprises culturing a genetically modified host cell as disclosed herein, in a medium containing levulinic acid, under conditions and for a time wherein at least a portion of the levulinic acid is catabolized by action of the host cell into 2-butanone.

Also disclosed herein is a method of inducing a host cell to make 2-butanone from levulinic acid. The method comprises introducing into the host cell a heterologous operon encoding genes whose encoded proteins enable the host cell to catabolize levulinic acid into 3-hydroxyvaleryl-CoA (3HV-CoA); upregulating expression of 3-hydroxyacyl-CoA dehydrogenase in the host cell to drive oxidation of at least a portion of 3HV-CoA to 3-ketovaleryl-CoA (3KV-CoA); and wherein the host cell expresses a nucleotide sequence encoding acetoacetyl-CoA transferase (atoDA) to drive conversion of at least a portion of the 3KV-CoA into 3-ketovalerate; and also wherein the host cell expresses a nucleotide sequence encoding acetoacetate decarboxylase (adc) to drive conversion of at least a portion of the 2-ketovalerate into butanone. Optionally, the host cell is acetyl-CoA synthetase (Acs)-negative, phosphotransacetylase (Pta)-negative, and/or acetate kinase (Ack)-negative.

The heterologous genes described herein for catabolizing LA and producing 2-butanone may be introduced into the host cell on a single vector containing all the necessary genes and promoters. Alternatively, the heterologous genes may be introduced into the host cell on several separate vectors and under the control of separate promoters. Thus, also disclosed herein is a combination of recombinant expression vectors, the combination comprising one or more expression vectors, each vector having one or more promoters operably linked to one or more genes selected from the group consisting of lvaA, lvaB, lvaC, lvaD, and lvaE. The combination may optionally include a nucleotide sequence encoding an acetoacetyl-CoA transferase, a short-chain thioesterase, and/or a succinyl-CoA transferase, operably linked to a promoter. These gene(s) and promoter(s) may be found in at least one of the vectors along with one or more of the other genes, or may be present in one or more additional vectors. Optionally, at least one of the vectors, or an additional vector, comprises a nucleotide sequence encoding an acetoacetate decarboxylase and which is operably linked to a promoter. Also optionally, at least one of the vectors, or one or more additional vectors, comprises a nucleotide sequence encoding FadB and/or FadJ, both of which nucleotide sequences are operably linked to one or more corresponding promoters.

### BRIEF DESCRIPTION OF THE DRAWINGS

FIG. 1 depicts P. putida lva operon genetic characterization. Panel A: Organization of the lvaRABCDEFG (9,323 bp) operon. Panel B: Reverse Transcriptase (RT) PCR demonstrates that each gene is expressed in cells grown on LA. Samples were compared with the negative control (−RT) where reverse transcriptase was omitted from the reaction. Panel C: RT-PCR of cDNA created with primer JMR237 demonstrates that the operon is polycistronic. Note that a product spanning each intergenic region was observed. Panel D: The transcription start sites (TSS) of regulator lvaR and lvaA were identified by 5′-RACE. Underlined sequence indicates ATG start codon. Triangle highlights experimentally determined TSS. Boxed sequence indicates previously annotated translation start site for lvaA.

FIG. 2A is schematic of transcriptional GFP fusion used to test induction of the lva operon. lvaR was cloned onto a plasmid containing its native constitutive promoter and the native promoter region for lvaA. The fluorescent protein sfGFP was cloned in place of lvaA.

FIG. 2B depicts the results of a lva operon induction assay. GFP fluorescence was measured from LB-cultures supplemented with various organic acids (20 mM) (n=3). Error bars represent standard deviation.

FIG. 3 depicts the proposed pathway for LA metabolism. LA, levulinic acid; 4HV, 4-hydroxyvalerate; 3HV, 3-hydroxyvalerate; LA-CoA, levulinyl-CoA; 4HV-CoA, 4-hydroxyvaleryl-CoA; CoA, coenzyme-A; ATP, adenosine triphosphate; 4PV-CoA, 4-phosphovaleryl-CoA; 3KV-CoA, 3-ketovaleryl-CoA; NAD(P)H, Nicotinamide adenine dinucleotide (phosphate) reduced.

FIG. 4A is a schematic of CoA-ligase activity assay. Using the Enzchek®-brand Pyrophosphatase Assay kit, the amount of pyrophosphate released during the CoA ligase reaction was measured as an increase of absorbance at 360 nm.

FIG. 4B depicts the results of the LvaE CoA-Ligase activity towards a variety of short and medium chain acids (n=3). Baseline subtraction was performed on all samples with a control reaction containing no substrate, indicated by Δ absorbance. Error bars represent standard deviation.

FIG. 5A and FIG. 5B depict CoA species abundance in LC/MS analysis of in vitro enzyme combinations. FIG. 5A is a histogram depicting the abundance of CoA species created after 30 min of incubating LA, ATP, NAD(P)H with varying enzyme combinations (n=3). ABDE-C indicates that the LvaABDE reaction was performed first, metabolites were separated from LvaABDE, and the resulting solutions were supplemented with LvaC solely. The reaction confirms that LvaC is capable of converting 4PV-CoA to 3HV-CoA. FIG. 5B is a plot depicting the abundance of CoA species over a 60 minute time course for a mixture of LvaABCDE, LA, ATP, and NAD(P)H (n=3). Error bars represent standard deviation.

FIGS. 6A, 6B, 6C, and 6D are comparisons of 4HV-CoA and 4PV-CoA MS/MS spectra. FIG. 6A: MS/MS spectra for 4HV-CoA. FIG. 6B: Assignment of selected fragments from 4HV-CoA. FIG. 6C: MS/MS spectra for 4PV-CoA. FIG. 6D: Assignments of selected fragments from 4PV-CoA. The masses between the selected fragments of 4PV-CoA and 4HV-CoA differ by the mass of PO₃H⁻ (79.967), indicating 4PV-CoA contains a phosphate group not found in 4HV-COA. Bold values indicate the mass of the parent ion. Peaks identified with the symbol (*) are fragments resulting from coenzyme A.

FIG. 7A is a representation of lva operon enzymatic genes. FIG. 7B is a comparison of LA degradation gene clusters found in other organisms.

FIG. 8 is a graph showing E. coli MG1655 growth with propionate using minimal media supplemented with propionate or propionate and acetate.

FIG. 9 is a growth curve of LS5218 strains on LA.

FIG. 10 depicts the production pathway for 2-butanone.

FIG. 11 is a histogram showing Acetone and butanone production from eMEK1 containing lvaABCDE and adc. Media consists of LB supplemented with LA, acetate or LA and acetate. (n=1).

FIG. 12 shows the optimized 2-butanone production with competing thiolase pathways deleted (strain eMEK4).

FIG. 13A shows acetone and butanone production from eMEK4 containing lvaABCDE and adc. FIG. 13B shows butanone yield from LA consumed. Media consisted of LB supplemented with acetate or LA and acetate.

FIG. 14 depicts growth coupling of acetate utilization and MEK production.

FIG. 15A, 15B, 15C, and 15D depict predicted phase planes from metabolic modeling for MEK production. FIG. 15A shows growth on LA. FIG. 15B shows growth on LA and acedate. FIG. 15C shows growth on LA for ΔatoB, and FIG. 15D shows growth of Δacs ΔackApta on LA and acetate.

FIG. 16 depicts the proposed pathway for GVL degradation through ring opening via lactonase activity. 4HV, 4-hydroxyvalerate; GVL, γ-valerolactone.

OPERON SEQUENCES
lvaA
nt:
(SEQ. ID. NO: 1)
atgagcagttcaccaacgatttccccggccagcgatacgttcgcggc
catgactgacgatcaccgcctggccgagttcatccgcgagcaggcct
cggcaacgcgggtggtcatccaggcgcgcaagcgcctgagcggcggc
gctatccaggaaaactggctgctggacctgctgatcgaaggcggccc
gtgggccggtgtccggcgttgggtactgcgcagcgatgcgctttcag
cgctacccgccagccttgaccgtgaacaggagttcgccgtgctgcag
gtggtttaccaggccggcgtgaaagtgccacgcccgctctggctgtg
ccgcgatgtgcgcgtgcatgggcgggtgttcttcctgatggagtatg
tgccgggtagcgctgccggccgcgcgctcagcaccggcgccggtcct
cagggccgggcgcaactggcgacgcagcttggcgccaacctggcgcg
tctgcatcaggtccgcccgccgtgcgccacgctgtgcttcctgtccg
ttccggacagctcgccggccctggcgaccatcgacgcctaccgccgc
tacctcgacaccctcgccgatgcctatccggtgctggaatggggcct
gcgctggtgcgagctgcatgcgccgcgcagcagcaccctgtgcctgt
tgcaccgtgactaccgcaccggcaactacctggccagcgaagaaggg
ctggaggccgtgctcgactgggagttcaccggctggggagatccttg
cgaggacctcggctggttcaccgcccgttgctggcgttttacccgtc
cagacctcgaagccggcggcattggccagctggaggattttctgcgt
ggttatcacgaggtgtcttcgctgtgcatcgagcgcagtcggctcca
ctactggcaagtcatggccaccctgcgctgggcggtgattgccttgc
agcaagggcagcgccatctgtccggtgaagaaccgtcgctcgagcta
gcactgacagcccggctgttgccggagctcgaactcgacatcctgca
catgaccggagccgaagcgccatga
aa:
(SEQ. ID. NO: 2)
MSSSPTISPASDTFAAMTDDHRLAEFIREQASATRVVIQARKRLSGG
AIQENWLLDLLIEGGPWAGVRRWVLRSDALSALPASLDREQEFAVLQ
VVYQAGVKVPRPLWLCRDVRVHGRVFFLMEYVPGSAAGRALSTGAGP
QGRAQLATQLGANLARLHQVRPPCATLCFLSVPDSSPALATIDAYRR
YLDTLADAYPVLEWGLRWCELHAPRSSTLCLLHRDYRTGNYLASEEG
LEAVLDWEFTGWGDPCEDLGWFTARCWRFTRPDLEAGGIGQLEDFLR
GYHEVSSLCIERSRLHYWQVMATLRWAVIALQQGQRHLSGEEPSLEL
ALTARLLPELELDILHMTGAEAP*
lvaB
nt:
(SEQ. ID. NO: 3)
atgacccaacccaacgcccacgaattgctcgagatcgcccgcgcgac
gcttctggagcagctgctgccagcgctgcccggcgagttgcgttacc
cggccctgatgatcgccaacgccatggccattgcggcccgcgaaaac
cgcttgggcgctcaggccgaggatcaggagcaggcgcgtctggccgc
cttggtcgatgacgcgccgtcgacattgcccgacctgcgccgccaac
tggctcgcgccattcgccagggcagccatgacgccccgcaaacccgg
cgcaccctggtcgagacattacgccagatcaccgttgcccgattggc
gatcagcaaccccaaggccttgccctga
aa:
(SEQ. ID. NO: 4)
MTQPNAHELLEIARATLLEQLLPALPGELRYPALMIANAMAIAARE
NRLGAQAEDQEQARLAALVDDAPSTLPDLRRQLARAIRQGSHDAPQ
TRRTLVETLRQITVARLAISNPKALP*
lvaC
nt:
(SEQ. ID. NO: 5)
atgaacttcactctcccggacgaactgctcgccttgcaggccaaga
ctcgagacttcattgccgaacaggtcatcccattcgagaacgaccc
ccgccagaacagccacggccccagcgacgcactgcgccaggacctg
gtgctctgcgcccgcgccgctggcttgctgacgcctcacgccagcc
gcgaaatgggcggtctggaactgagccatgtggccaaggcgatcgt
cttcgaagaagccggctactcgccgctgggcccggtagcgctgaat
atccatgcgccggacgaaggcaatatccacctgatggacgtggtcg
ccaccgaagcgcagaaggaccgctggttgcgcccgctggtccaggg
ccatgcccgttcgtgcttcgccatgacggagcctgctccgggctcc
ggttcggatccgtcgatgctgcgcaccactgccacccgcgatggcg
acgactacctgatcaatggtcgcaagtggctgatcaccggggccga
aggcgcggacttcggcatcatcatggcgcgcatggaggacggcacc
gcgaccatgttcctgaccgacatgaagcgcgacggcatcatccatg
aacgtcagctggactcgctggacagctgttttaccggcggtcacgg
gcagctgcgtttcgacaacctgcgtattccggcgagcgatgtcctc
ggcgagatcggcaagggcttccggtatgcccaggtgcgcctggcgc
ctgcacgcttgactcattgcatgcgctggctcggtgccgcgcgccg
cgcccacgacatcgcctgcgactatgcgcgcacccgggacgccttt
ggcaagccgctgggcgagcaccagggcgtgggtttcatgctggccg
acaacatgatggacctgcacgtggtgcgtctggcggtctggcactg
cgcctgggtgctcgaccagggccggcgcgccaatgtcgattcgagc
atggccaaggtgatcagcgccgaggcgctgtggcgggtggtcgatc
gttgcgtccaggtattgggtggacgcggggtgaccggggacaccgt
ggtggagcggatcttccgcgacattcgcccgttccgcatctatgac
ggcccgagcgaagtgcaccgcatgagcctggcgaagaagctgctcg
accagcgcctggaggcccactga
aa:
(SEQ. ID. NO: 6)
MNFTLPDELLALQAKTRDFIAEQVIPFENDPRQNSHGPSDALRQDL
VLCARAAGLLTPHASREMGGLELSHVAKAIVFEEAGYSPLGPVALN
IHAPDEGNIHLMDVVATEAQKDRWLRPLVQGHARSCFAMTEPAPGS
GSDPSMLRTTATRDGDDYLINGRKWLITGAEGADFGIIMARMEDGT
ATMFLTDMKRDGIIHERQLDSLDSCFTGGHGQLRFDNLRIPASDVL
GEIGKGFRYAQVRLAPARLTHCMRWLGAARRAHDIACDYARTRDAF
GKPLGEHQGVGFMLADNMMDLHVVRLAVWHCAWVLDQGRRANVDSS
MAKVISAEALWRVVDRCVQVLGGRGVTGDTVVERIFRDIRPFRIYD
GPSEVHRMSLAKKLLDQRLEAH*
lvaD
nt:
(SEQ. ID. NO: 7)
atgcagccgaaccttgcccgactgttcgccctcgacgggcgtcgc
gccctggtgaccggggcctccagcggcctgggccgtcacttcgcc
atgaccctggccgccgcaggcgccgaggtggtggtgaccgccaga
cgccaggcgccgctgcaggcgttggtggaggccatcgaggtggcc
ggagggcgggcgcaggcctttgccctcgatgtgacgagccgtgag
gacatctgccgggtgctcgatgccgccggcccgctggatgttctg
gtcaacaatgcgggggtgagcgacagccagcctttgctagcctgc
gatgatcaaacctgggaccacgtgctcgacaccaacctcaagggc
gcctgggccgtggcccaggaaagcgcccggcgcatggtggtggcg
gggaaggggggcagcctgatcaatgtcacctcgatcctcgccagc
cgtgtggccggcgccgtcggcccttacctggcggccaaggccggc
ctggcccacctgacccgcgccatggcgctggagttggcgcgccat
ggtatccgggtgaacgccctggcgcccggctacgtgatgactgat
ttgaacgaggccttcctggccagcgaggccggtgacaagttgcgc
tcgcggatccccagccgccgcttcagcgtgccgtcggacctggac
ggcgccttgctgctgctcgccagcgatgccgggcgggcgatgagc
ggcgctgagatcgtggtcgatggcggccacctgtgcagcagcctg
taa
aa:
(SEQ. ID. NO: 8)
MQPNLARLFALDGRRALVTGASSGLGRHFAMTLAAAGAEVVVTAR
RQAPLQALVEAIEVAGGRAQAFALDVTSREDICRVLDAAGPLDVL
VNNAGVSDSQPLLACDDQTWDHVLDTNLKGAWAVAQESARRMVVA
GKGGSLINVTSILASRVAGAVGPYLAAKAGLAHLTRAMALELARH
GIRVNALAPGYVMTDLNEAFLASEAGDKLRSRIPSRRFSVPSDLD
GALLLLASDAGRAMSGAEIVVDGGHLCSSL*
lvaE
nt:
(SEQ. ID. NO: 9)
atgatggttccaaccctcgaacacgagcttgctcccaacgaagcc
aaccatgtcccgctgtcgccgctgtcgttcctcaagcgtgccgcg
caggtgtacccgcagcgcgatgcggtgatctatggcgcaaggcgc
tacagctaccgtcagttgcacgagcgcagccgcgccctggccagt
gccttggagcgggtcggtgttcagccgggcgagcgggtggcgata
ttggcgccgaacatcccggaaatgctcgaggcccactatggcgtg
cccggtgccggggcggtgctggtgtgcatcaacatccgcctggag
gggcgcagcattgccttcatcctgcgtcactgcgcggccaaggta
ttgatctgcgatcgtgagttcggtgccgtggccaatcaggcgctg
gccatgctcgatgcgccgcccttgctggtgggcatcgacgatgat
caggccgagcgcgccgatttggcccacgacctggactacgaagcg
ttcttggcccagggcgaccccgcgcggccgttgagtgcgccacag
aacgaatggcagtcgatcgccatcaactacacctccggcaccacg
ggggaccccaagggcgtggtgctgcatcaccgcggcgcctacctc
aacgcctgcgccggggcgctgatcttccagttggggccgcgcagc
gtctacttgtggaccttgccgatgttccactgcaacggctggagc
catacctgggcggtgacgttgtccggtggcacccacgtgtgtctg
cgcaaggtccagcctgatgcgatcaacgccgccatcgccgagcat
gccgtgactcacctgagcgccgccccagtggtgatgtcgatgctg
atccacgccgagcatgccagcgcccctccggtgccggtttcggtg
atcactggcggtgccgccccgcccagtgcggtcatcgcggcgatg
gaggcgcgtggcttcaacatcacccatgcctatggcatgaccgaa
agctacggtcccagcacattgtgcctgtggcagccgggtgtcgac
gagttgccgctggaggcccgggcccagttcatgagccgccagggc
gtcgcccacccgctgctcgaggaggccacggtgctggataccgac
accggccgcccggtcccggccgacggccttaccctcggcgagctg
gtggtgcggggcaacactgtgatgaaaggctacctgcacaaccca
gaggctacccgtgccgcgttggccaacggctggctgcacacgggc
gacctggccgtgctgcacctggacggctatgtggaaatcaaggac
cgagccaaggacatcatcatttctggcggcgagaacatcagttcg
ctggagatagaagaagtgctctaccagcaccccgaggtggtcgag
gctgcggtggtggcgcgtccggattcgcgctggggcgagacacct
cacgctttcgtcacgctgcgcgctgatgcactggccagcggggac
gacctggtccgctggtgccgtgagcgtctggcgcacttcaaggcg
ccgcgccatgtgtcgctcgtggacctgcccaagaccgccactgga
aaaatacagaagttcgtcctgcgtgagtgggcccggcaacaggag
gcgcagatcgccgacgccgagcattga
aa:
(SEQ. ID. NO: 10)
MMVPTLEHELAPNEANHVPLSPLSFLKRAAQVYPQRDAVIYGARR
YSYRQLHERSRALASALERVGVQPGERVAILAPNIPEMLEAHYGV
PGAGAVLVCINIRLEGRSIAFILRHCAAKVLICDREFGAVANQAL
AMLDAPPLLVGIDDDQAERADLAHDLDYEAFLAQGDPARPLSAPQ
NEWQSIAINYTSGTTGDPKGVVLHHRGAYLNACAGALIFQLGPRS
VYLWTLPMFHCNGWSHTWAVTLSGGTHVCLRKVQPDAINAAIAEH
AVTHLSAAPVVMSMLIHAEHASAPPVPVSVITGGAAPPSAVIAAM
EARGFNITHAYGMTESYGPSTLCLWQPGVDELPLEARAQFMSRQG
VAHPLLEEATVLDTDTGRPVPADGLTLGELVVRGNTVMKGYLHNP
EATRAALANGWLHTGDLAVLHLDGYVEIKDRAKDIIISGGENISS
LEIEEVLYQHPEVVEAAVVARPDSRWGETPHAFVTLRADALASGD
DLVRWCRERLAHFKAPRHVSLVDLPKTATGKIQKFVLREWARQQE
AQIADAEH*

### DETAILED DESCRIPTION

### Abbreviations and Definitions

ATP=adenosine triphosphate. CoA=coenzyme-A. 3HV=3-hydroxyvalerate. 4HV=4-hydroxyvalerate. 4HV-CoA=4-hydroxyvaleryl-CoA. 3KV-CoA=3-ketovaleryl-CoA. LA=levulinic acid. LA-CoA=levulinyl-CoA. MOPS=3-(N-morpholino) propanesulfonic acid. NAD(P)H=Nicotinamide adenine dinucleotide (phosphate) reduced. 4PV-CoA=4-phosphovaleryl-CoA. 5′-RACE-Rapid Amplification of cDNA Ends. sfGFP=super-folder green fluorescent protein (see Pédelacq J D, Cabantous S, Tran T, Terwilliger T C, Waldo G S, Nat Biotechnol. 2006 Jan; 24(1):79-88). TSS=transcription start site.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (also called “non-coding” RNA or “ncRNA”; e.g. tRNA, rRNA, a ribozyme, etc.).

A “protein coding sequence” or “coding region” is a sequence that encodes a particular protein or polypeptide. A “protein coding sequence” or “coding region” is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic, viral, or eukaryotic mRNA, genomic DNA sequences from prokaryotic, viral, or eukaryotic DNA, and synthetic nucleic acids. A transcription termination sequence will usually be located 3′ to the coding region.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different manners. To determine sequence identity, sequences can be aligned using various methods and computer programs (e.g., BLAST, T-COFFEE, MUSCLE, MAFFT, etc.), all of which are well known in the art. “BLAST”=Basic Local Alignment Search Tool; available online from the U.S. National Library of Medicine at https://blast.ncbi.nlm.nih.gov/Blast.cgi; see Altschul, Gish, Miller, Myers, and Lipman, (1990) “Basic local alignment search tool,” Journal of Molecular Biology. 215(3):403-410. “T-COFFEE”=Tree-based Consistency Objective Function for Alignment Evaluation; see Notredame C, Higgins D G, Heringa J (2000 Sep 8) “T-Coffee: A novel method for fast and accurate multiple sequence alignment,” J Mol Biol 302(1):205-217; available online at http://tcoffee.org/. “MUSCLE”=Multiple Sequence Comparison by Log-Expectation; available online from the European Bioinformatics Institute (EMBL-EBI) at https://www.ebi.ac.uk/Tools/msa/muscle/; see Edgar, RC (2004) “MUSCLE: a multiple sequence alignment method with reduced time and space complexity,” BMC Bioinformatics, 5:113. “MAFFT”=Multiple Alignment using Fast Fourier Transform; available online at https://mafft.cbrc.jp/alignment/software/; see Katoh, Misawa, Kuma, Miyata (2002) “MAFFT: a novel method for rapid multiple sequence alignment based on fast Fourier transform,” Nucleic Acids Res. 30:3059-3066.

The term “binding”, as used herein refers to a non-covalent interaction between macromolecules (e.g., between a protein and a nucleic acid). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (Kd) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower Kd.

A “promoter sequence” is a DNA regulatory region capable of binding RNA polymerase and initiating transcription of a downstream (3′ direction) coding or non-coding sequence. For purposes of defining the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various prokaryotic and eukaryotic promoters, including inducible promoters, may be used in the various recombinant expression vectors of the present disclosure. The promoter may be a constitutively active promoter, i.e. a promoter that is active in the absence externally applied agents, or it may be an inducible promoter (e.g., T7 RNA polymerase promoter, T3 RNA polymerase promoter, Isopropyl-beta-D-thiogalactopyranoside (IPTG)-regulated promoter, heat shock promoter, anhydro tetracycline-regulated promoter, arabinose-inducible promoter, CRISPRi-regulated promoter, TAL-Effector-regulated promoter, phosphate-starvation-regulated promoter, steroid-regulated promoter, metal-regulated promoter, methionine-inducible promoter; a galactose-inducible promoter, and the like). As used herein, an inducible promoter is a promoter whose activity is regulated upon the application of an agent to the cell, (e.g. doxycycline) or the induced presence of a particular RNA polymerase (e.g., T7 RNA polymerase).

Agents that induce any given inducible promoter are known in art. For example, tetracycline-regulatable promoters can be regulated by tetracycline or doxycycline; carbohydrates can be used to induce a carbohydrate-inducible promoter (e.g., galactose for a galactose-inducible promoter); methionine can be used to induce a methionine-inducible promoter; metals can be used to induce a metallothionein promoter, etc.).

The terms “control element,” and “regulatory element,” used interchangeably herein, refer to transcriptional, translational, and degradation control sequences that are transcribed as part of the RNA molecule whose activity that they regulate. Such regulatory elements can control a wide variety of processes (activities) including but not limited to transcription (e.g., initiation, elongation, and/or termination), translation (initiation, elongation, and/or termination), RNA stability, etc. Regulatory elements include but are not limited to recognition sequences for antisense RNAs, leader sequences, riboswitches, a 5′ methyl cap, a 3′ poly-A tail, sequences recognized by ribozymes, sequences recognized by ribosomes (e.g., a ribosome binding site (RBS), e.g., Shine-Delgarno Sequence), self-cleaving ribozymes, leader-sequences, sequences bound by RNA binding proteins, sequences targeted by a guide-strand-bound RISC complex, etc.

Some regulatory elements are operably linked to a promoter, but reciprocally regulate transcription (e.g., via early termination of RNA polymerase elongation) such that the promoter affects transcription of the regulatory element and the regulatory element also affects transcription of its own transcript. Some regulatory elements (e.g., IS10wt, IS10-9, and others known in the art: the RNA-IN/OUT translation control system) can function as part of an antisense RNA-mediated translation control system (Mutalik et al. Nature Chem. Biol. 2012 (8) May: 447-454; Kittle et al. J Mol. Biol. 1989 Dec. 5; 210 (3): 561-72: Insertion sequence IS10 anti-sense pairing initiates by an interaction between the 5′ end of the target RNA and a loop in the anti-sense RNA). Other exemplary regulatory elements that find use in the expression vectors, compositions, methods, and kits of this disclosure include but are not limited to PT181 wt and its orthologs, IS10wt and its orthologs, Bujard RBS, B0030 RBS, Weiss RBS, Anderson RBS, lacZp1 UTR, serB UTR, chiA UTR, lacY UTR, sodA UTR, ompRp3UTR, trpR UTR, glpA UTR, rhoL UTR, CRISPRI UTR, fixA UTR, lldP UTR, and the like.

The term “naturally-occurring” or “unmodified” as used herein as applied to a nucleic acid, a polypeptide, a cell, or an organism that is found in nature.

“Recombinant” means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame and may indeed act to modulate production of a desired product by various mechanisms (see “regulatory element”, above). Alternatively, DNA sequences encoding RNA that is not translated may also be considered recombinant. Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant” polypeptide refers to a polypeptide which is not naturally occurring, e.g., is made by the artificial combination of two otherwise separated segments of amino sequence through human intervention.

A “vector” or “expression vector” is a replicon, such as plasmid, phage, virus, or cosmid, to which another DNA segment (an “insert”) may be attached so as to bring about the replication of the attached segment in a cell. An “expression cassette” comprises a DNA coding sequence operably linked to a promoter.

The term “operably linked” refers to a physical juxtaposition of nucleic acids in a polynucleotide wherein the components so described are in a relationship permitting them to function in their intended manner. For instance, a coding sequence is operably linked to a promoter (or the promoter can be said to be operably linked to the coding sequence) if the promoter affects the transcription or expression of the coding sequence. If a regulatory element is operably linked to a promoter, the regulatory element is transcribed and the promoter affects the transcription of the regulatory element. Nucleotides that are operably linked need not be (and often are not) directly linked to each other.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence.

A recombinant expression vector may also contain an insertion site for the insertion of a sequence of interest. An “insertion site” is any nucleotide sequence intentionally positioned within the vector that allows for convenient insertion and/or excision of additional nucleic acid sequences. The term “insertion site” encompasses sequences that facilitate any convenient cloning methodology (e.g., standard restriction enzyme/ligation based methods, integrase based methods, T4 DNA Polymerase based methods, BioBrick cloning, Circular Polymerase Extension Cloning (CPEC) cloning, etc.) (Quan, J. & Tian, J. Nat. Protoc. 6, 242-251 (2011); Shetty et al. J. Biol. Eng. 2, 5 (2008)). An example of one possible type of standard insertion site is a multiple cloning site (or polylinker), which is a stretch of sequences that contains multiple restriction enzyme sites that together facilitate convenient restriction enzyme/ligation based cloning methods.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include viral infection, transfection, conjugation, protoplast fusion, electroporation, particle gun technology, calcium phosphate precipitation, direct micro injection, and the like. The choice of method is generally dependent on the type of cell being transformed and the circumstances under which the transformation is taking place (in vitro, ex vivo, or in vivo). A general discussion of these methods can be found in Ausubel, et al., Short Protocols in Molecular Biology, 5th ed., Wiley & Sons, 2002.

Disclosed herein are a series of isolated genes and their corresponding encoded proteins. The genes have been assigned the names lvaA, lvaB, lvaC, lvaD, and lvaE. The corresponding proteins encoded by the genes have been assigned the names LvaA, LvaB, LvaC, LvaD, and LvaE. The genes and the proteins encoded thereby are explicitly defined herein as follows:

lvaA is defined as a gene encoding a phosphotransferase that phosphorylates the 4-hydroxy position on 4-hydroxyvaleryl-CoA (4HV-CoA) to form 4-phosphovaleryl-CoA (4PV-CoA). The gene has at least 80% sequence identity and more preferably at least 90% sequence identity to PP_2791 from Psuedomonas putida.

lvaB is defined as a gene encoding a small protein associated with LvaA that is essential for the phosphorylation of 4HV-CoA by LvaA. The gene has at least 80% sequence identity and more preferably at least 90% sequence identity to PP_2792 from Psuedomonas putida. In some cases, a single protein contains sequence homology to both LvaA and LvaB. (That is, LvaA and LvaB appear as a type of fusion protein.)

lvaC is defined as a gene encoding an acyl-CoA dehydrogenase family protein that hydrates either 2-pentenoyl-CoA or 3-pentenoyl-CoA to form 3-hydroxyvaleryl-CoA (3HV-CoA). The gene has at least 80% sequence identity and more preferably at least 90% sequence identity to PP_2793 from Psuedomonas putida.

lvaD is defined as a gene encoding a reductase that reduces 4-ketovaleryl-CoA to 4HV-CoA. The gene has at least 80% sequence identity and more preferably at least 90% sequence identity to PP_2794 from Psuedomonas putida.

lvaE is defined as a gene encoding a protein that acts as a acyl-CoA synthetase on levulinic acid to form levulinyl-CoA (4-ketovaleryl-CoA). The gene has at least 80% sequence identity and more preferably at least 90% sequence identity to PP_2795 from Psuedomonas putida.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present invention shall include the corresponding plural characteristic or limitation, and vice-versa, unless otherwise specified or clearly implied to the contrary by the context in which the reference is made. The singular indefinite articles “a” and “an” mean “one or more,” unless specifically defined otherwise.

All combinations of method or process steps as used herein can be performed in any order, unless otherwise specified or clearly implied to the contrary by the context in which the referenced combination is made.

The methods and genetic constructs of the present invention can comprise, consist of, or consist essentially of the essential elements and limitations as described herein, as well as any additional or optional ingredients, components, or limitations described herein or otherwise useful in recombinant genetics.

### Identification of Genes Involved in Levulinic Acid Metabolism

P. putida KT2440 is known to metabolize LA as a sole carbon source and demonstrates diauxic growth in the presence of glucose and LA. Therefore, a genetic study was initiated to identify genes involved in LA catabolism. A mutant library was constructed with a Tn5 mini transposase (Martínez-García, E., Calles, B., Arévalo-Rodríguez, M. & de Lorenzo, V. pBAM1: an all-synthetic genetic tool for analysis and construction of complex bacterial phenotypes. BMC Microbiol. 11, 38 (2011)) and screened for P. putida mutants lacking the ability to grow on LA as the sole carbon source. Thirteen out of 7,000 colonies screened demonstrated LA growth deficiencies. The location of each transposon insertion was determined by sequencing PCR products created with a primer nested in the transposon paired with a degenerate random primer. Table 1 shows the ten unique isolates from these thirteen hits and the putative function of the disrupted genes. Two mutants had disruptions in genes involved in propionate metabolism, supporting the hypothesis that LA is catabolized to the central metabolites, acetyl-CoA and propionyl-CoA. Three transposon mutants had disruptions in a putative operon that had not been previously characterized (disrupting genes PP_2791, PP_2793, and PP_2794). Other mutants had disruptions in genes with no obvious connection to LA catabolism (bioH, gcvP, a hypothetical zinc protease, mrdA, and fpvA). To confirm that a sufficient number of clones had been screened, a random bar code transposon-site sequencing (RB-TnSeq) was performed for cultures enriched by growth on LA and 4HV relative to growth on glucose. RB-TnSeq is an efficient method for determining gene essentiality under different conditions with high genomic coverage. Wetmore, K. M. M. et al. Rapid Quantification of Mutant Fitness in Diverse Bacteria by Sequencing Randomly Bar-Coded Transposons. MBio 6, 1-15 (2015). This analysis identified additional genes involved in LA metabolism including an acetoacetyl-CoA transferase important for growth on LA, genes functioning in β-oxidation and propionyl-CoA metabolism, and 14 transcriptional regulators potentially involved in LA metabolism. The RB-TnSeq dataset also revealed that 3-hydroxybutyryl-CoA dehydrogenase and β-ketothiolase are also necessary for growth on LA and 4HV, supporting our hypothesis that LA metabolism terminates through β-oxidation. For a more complete summary and analysis of the fitness data, see the Examples.

TABLE 1
P. putida Levulinic Acid Transposon Insertion Sites

| | Insertion | Gene | |
|---|---|---|---|
| Locus | Point* | Name | Description/Homology |
| PP_0364 | 442685 | bioH | pimeloyl-ACP methyl ester esterase |
| PP_0988 | 1128706 | gcvP-1 | glycine dehydrogenase |
| PP_2332 | 2660666 | N/A | ATP-dependent zinc protease family |
| PP_2336 | 2666405 | acnA-II | aconitate hydratase |
| PP_2337 | 2666944 | prpF | aconitate isomerase |
| PP_2791 | 3181098 | N/A | Phosphotransferase family |
| PP_2793 | 3182533 | N/A | acyl-CoA dehydrogenase family protein |
| PP_2794 | 3183601 | N/A | short chain dehydrogenase/reductase |
| | | | family |
| PP_3741 | 4271628 | mrdA-I | transpeptidase |
| PP_4217 | 4765953 | fpvA | TonB-dependent outer membrane |
| | | | ferripyoverdine receptor |

*Insertion point based on location from P. putida KT2440 origin

### Operon Characterization and Induction

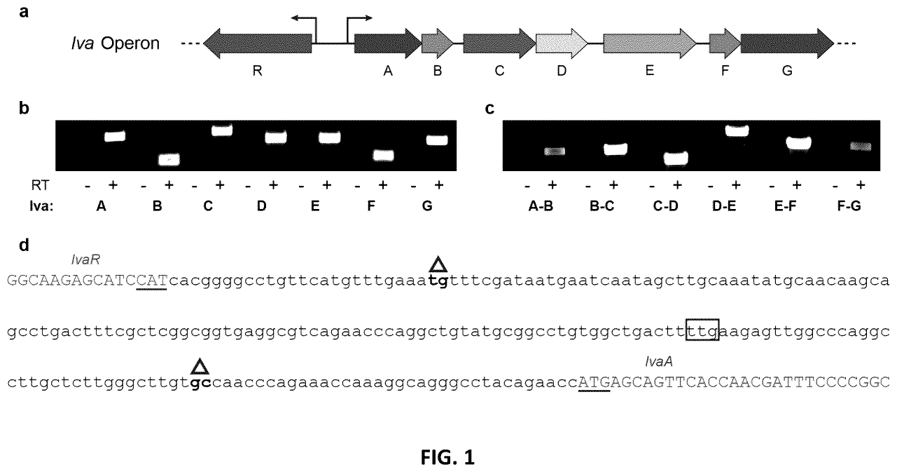

Given the propensity of bacteria to cluster related genes into operons, the putative seven-gene operon, PP_2791-PP_2797 was examined, which contained three of the transposon hits (PP_2791, PP_2793 and PP_2794). The sequence homology of the seven genes in the operon was analyzed using the basic local alignment search tool (BLAST; Altschul, S. F., Gish, W., Miller, W., Myers, E. W. & Lipman, D. J. Basic local alignment search tool. J. Mol. Biol. 215, 403-10 (1990)) and assigned predicted functions, which are listed in Table 2, below. There are no known published studies about these genes beyond the automated sequence annotations. Therefore, the expression and function of these genes was investigated to determine if they are involved in LA catabolism. First, RNA from wild type P. putida grown in minimal media with LA as the carbon source was isolated. All seven genes were then located by PCR amplification of cDNA created with a reverse primer specific to PP_2797. See FIG. 1, panels A, B, and C. The transcription start site (TSS) of the operon was isolated by 5′-RACE (see FIG. 1, panel D) and implicated a different start codon for PP_2791, 72 bp downstream of the one originally reported. Schramm, G., Bruchhaus, I. & Roeder, T. A simple and reliable 5′-RACE approach. Nucleic Acids Res 28, E96 (2000). Espah Borujeni, A., Channarasappa, A. S. S. & Salis, H. M. M. Translation rate is controlled by coupled trade-offs between site accessibility, selective RNA unfolding and sliding at upstream standby sites. Nucleic Acids Res. 42, 2646-2659 (2014). Salis, H. M. M., Mirsky, E. A. A. & Voigt, C. A. A. Automated design of synthetic ribosome binding sites to control protein expression. Nat. Biotechnol. 27, 946-50 (2009). A σ⁵⁴ promoter sequence located upstream of PP_2791 was identified by comparing upstream of the new TSS with published σ⁵⁴ promoter consensus sequences. Barrios, H., Valderrama, B. & Morett, E. Compilation and analysis of sigma(54)-dependent promoter sequences. Nucleic Acids Res. 27, 4305-4313 (1999). The data presented below suggests the proteins encoded by this operon are important in LA catabolism. The polycistronic genes are designated herein as lvaABCDEFG.

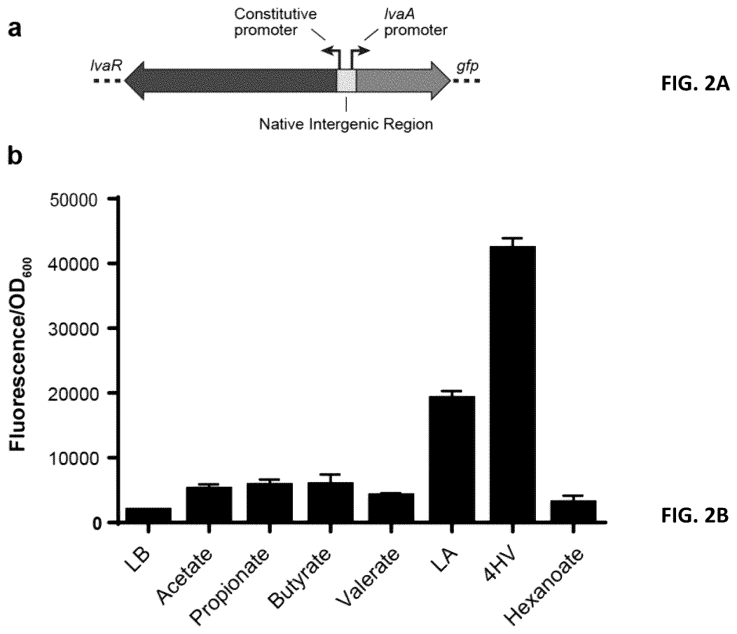

Upstream of lvaABCDEFG, a gene oriented divergently from the operon (PP_2790) was identified and predicted to encode a transcription factor with a σ⁵⁴ interaction domain and homology to the propionate metabolism activator, prpR. The genomic organization strongly suggested that the gene encoded a regulator for the lva operon. Consequently, PP_2790 was deleted and growth of P. putida strains was evaluated on both LA and a likely intermediate, 4HV. The APP_2790 mutant was unable to grow on LA and 4HV suggesting that it acts as an activator for the operon. Expression of PP_2790 on a plasmid restored growth of the deletion strain on LA and 4HV. To identify compounds that activate lvaABCDEFG expression, a transcriptional reporter system was built that linked sfGFP to the σ⁵⁴ promoter sequence located upstream of lvaA. The reporter cassette was cloned onto a broad host range vector (shown schematically in FIG. 2A) and the resulting construct was transformed into wild type P. putida. A variety of short and medium chain length acids were tested by adding them to rich media and evaluating the corresponding sfGFP expression levels. Strong sfGFP fluorescence was observed only when LA or 4HV were added to the system. See FIG. 2B. Without being limited to any underlying mechanism, it is thought that PP_2790 encodes a transcriptional regulator responsive to the LA pathway. It is designated herein as lvaR.

### Genetic and Biochemical Studies of lvaABCDEFG Operon

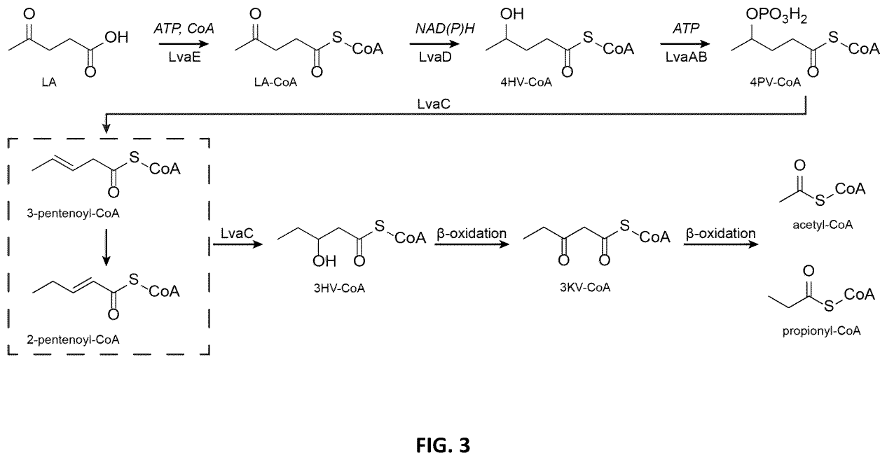

To confirm the involvement of the lva operon in LA catabolism, a deletion mutant was created for each lva gene predicted to encode an enzymatic protein and a corresponding complementation plasmid using the P_{araBAD} promoter. The ability of the resulting strains to grow on LA and 4HV was tested. See Table 2, below. In addition, we purified the five enzymes from cultures of E. coli BL21 (DE3), reconstituted the enzymatic reactions in vitro, and used liquid chromatography/mass spectrometry (LC/MS) to identify reaction products. Selective ion scanning was used to monitor the masses for likely intermediates based on prior studies. Jaremko, M. & Yu, J. The initial metabolic conversion of levulinic acid in Cupriavidus necator. J. Biotechnol. 155, 293-298 (2011). Martin, C. H. & Prather, K. L. J. High-titer production of monomeric hydroxyvalerates from levulinic acid in Pseudomonas putida. J. Biotechnol. 139, 61-67 (2009). Zhang, G. F. et al. Catabolism of 4-hydroxyacids and 4-hydroxynonenal via 4-hydroxy-4-phosphoacyl-CoAs. J. Biol. Chem. 284, 33521-33534 (2009). Harris, S. R. et al. Metabolism of levulinate in perfused rat livers and live rats: Conversion to the drug of abuse 4-hydroxypentanoate. J. Biol. Chem. 286, 5895-5904 (2011). The proposed pathway is shown in FIG. 3. First, LA is activated as a coenzyme A-thioester, levulinyl-CoA (LA-CoA). Second, LA-CoA is reduced to 4-hydroxyvaleryl-CoA (4HV-CoA). Third, 4HV-CoA is phosphorylated at the γ-position to yield 4-phosphovaleryl-CoA (4PV-CoA). Fourth, 4PV-CoA is dephosphorylated to yield a pentenoyl-CoA species (likely 3-pentenoyl-CoA). Last, pentenoyl-CoA is hydrated to yield 3-hydroxyvaleryl-CoA (3HV-CoA) which can be further oxidized via β-oxidation to yield acetyl-CoA and propionyl-CoA or incorporate 3HV-CoA into PHA polymers.

TABLE 2
P. putida LA Operon Knockout and Complementation

| | | Growth on LA | | Growth on 4HV | |
|---|---|---|---|---|---|
| Genotype | Predicted Function | EV | Complement | EV | Complement |
| WT | | ++ | N/A | ++ | N/A |
| ΔlvaR | σ⁵⁴ dependent | − | ++ | − | ++ |
| (PP_2790) | sensory box protein | | | | |
| ΔlvaA | Phosphotransferase | − | ++ | − | ++ |
| (PP_2791) | family | | | | |
| ΔlvaB | Hypothetical protein | − | ++ | − | ++ |
| (PP_2792) | | | | | |
| ΔlvaC | acyl-CoA dehydrogenase | − | ++ | + | ++ |
| (PP_2793) | family protein | | | | |
| ΔlvaD | short chain | − | ++ | ++ | ++ |
| (PP_2794) | dehydrogenase/reductase | | | | |
| | family | | | | |
| ΔlvaE | Acyl-CoA synthetase | ++ | ++ | − | + |
| (PP_2795) | | | | | |

(EV) empty vector plasmid;
(N/A) not applicable;
(−) No growth;
(+) Visible growth;
(++) Robust growth

### lvaE

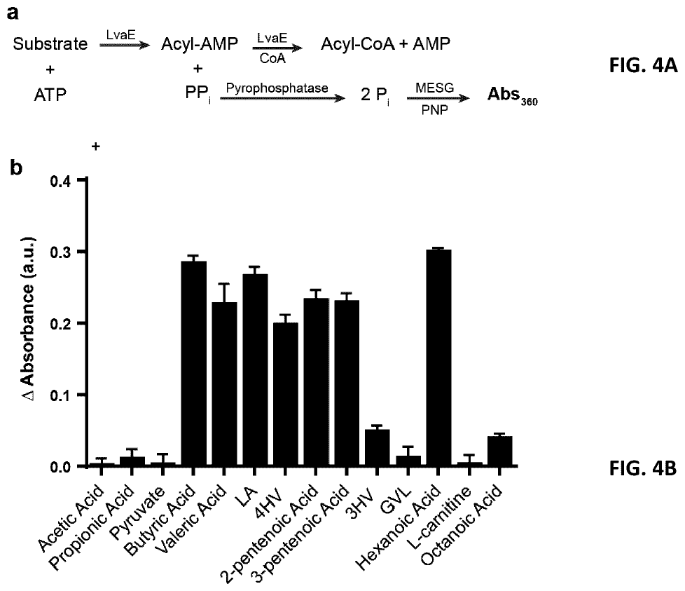

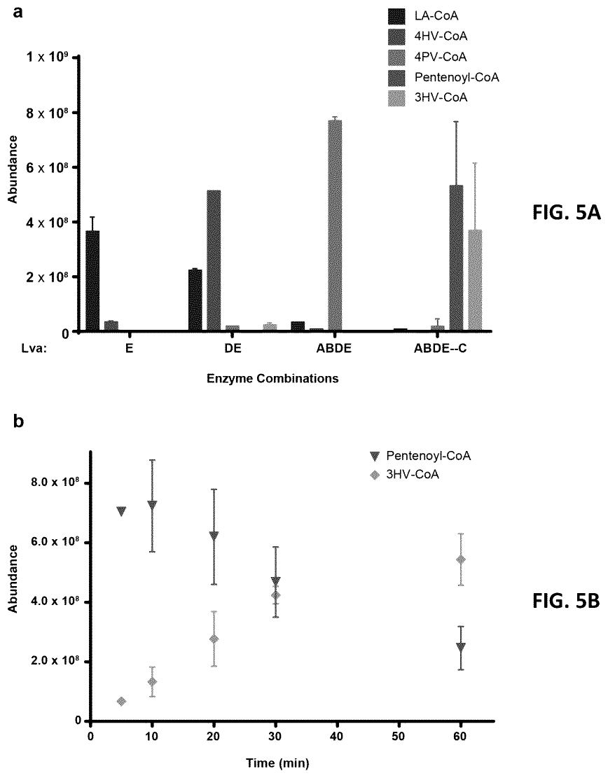

The presence of an enzyme (encoded by lvaE) with homology to an acyl-CoA synthetase (including a putative CoA binding region and an AMP binding site) suggested that the degradation pathway acts on CoA thioesters and begins with the activation of acids to acyl-CoA's. The ΔlvaE strain grew on LA but not on 4HV, indicating that LA may also be activated by other CoA-synthetases in P. putida. The activity of purified LvaE (6x-His N-terminal fusion) was quantified on a variety of organic acid substrates using the EnzChek®-brand Pyrophosphate Assay Kit (Molecular Probes, Eugene, OR) which detects pyrophosphate released in the first half reaction to creating the acyl-AMP intermediate. See FIG. 4A for a schematic. LvaE demonstrated activity on C₄-C₆ carboxylic acids, including LA and 4HV (see FIG. 4B), but showed minimal activity on other organic acids (pyruvate, acetate, propionate, octanoate). Using LC/MS to detect reaction products, it was demonstrated that LvaE was necessary and sufficient to catalyze the ligation of CoA to LA, generating levulinyl-CoA (LA-CoA). See FIGS. 5A and 5B. None of the other enzymes from the operon catalyzed this or any other reaction using LA as a substrate (data not shown), confirming that the pathway proceeds via acyl-CoA intermediates.

### lvaD

The second step in the proposed pathway is the reduction of LA-CoA to 4HV-CoA which is predicted to be catalyzed by lvaD. lvaD is annotated as an oxidoreductase containing an NADH binding domain and was found to be required for growth on LA but not necessary for growth on 4HV. See Table 2. LvaD was purified in a similar manner to LvaE but used an N-terminal maltose binding protein (MBP) tag to increase the solubility of the enzyme. Fox, J. D., Routzahn, K. M., Bucher, M. H. & Waugh, D. S. Maltodextrin-binding proteins from diverse bacteria and archaea are potent solubility enhancers. FEBS Lett. 537, 53-57 (2003). The in vitro reaction containing LvaD and LvaE verified that LvaD is involved in the production of 4HV-CoA. See FIG. 5A. Furthermore, LvaDE was the only enzyme combination capable of generating 4HV-CoA in vitro (data not shown). LvaD can catalyze the reduction of LA-CoA with either NADH or NADPH (data not shown).

### lvaAB

It was hypothesized that the third intermediate would be 4-phospho-valeryl-CoA (4PV-CoA) based off its observation in LA degradation in rat livers. (Zhang et al. and Harris et al., supra.) The first gene in the operon, lvaA, has putative homology regions, including an ATP binding site that associated it with the kinase superfamily and phosphotransferase family of enzymes. The second protein in the operon (LvaB) has no listed function and is predicted to be only 12 kDa in size. Orthologous sequence alignments of lvaB reveal that in all other organisms this hypothetical protein is located immediately downstream of an lvaA ortholog. Therefore, a pull down experiment was used to determine if the two proteins interact. Striebel, F. et al. Bacterial ubiquitin-like modifier Pup is deamidated and conjugated to substrates by distinct but homologous enzymes. Nat. Struct. Mol. Biol. 16, 647-651 (2009). Yamamoto, S. & Kutsukake, K. FliT acts as an anti-FlhD2C2 factor in the transcriptional control of the flagellar regulon in Salmonella enterica serovar typhimurium. J. Bacteriol. 188, 6703-8 (2006).

LvaA was N-terminally tagged with MBP and cloned into a pET expression vector. LvaB was cloned directly downstream of LvaA as it is found in P. putida's native genome sequence. The recombinant proteins were expressed in E. coli BL21 (DE3) and purified using the MBP tag. A SDS-page gel of the eluent contained two bands at 85 kDa and 12 kDa, closely matching the predicted sizes of MBP-LvaA and untagged LvaB respectively. Liquid chromatography tandem mass spectrometry was done on a trypsin digest of the 12 kDa band and identified the protein sequence to be LvaB (data not shown).

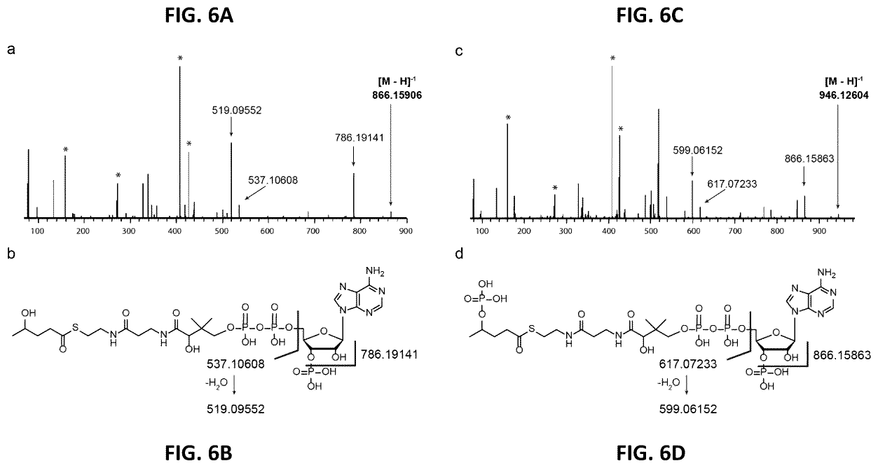

Growth studies of deletion mutants revealed that lvaA and lvaB are both required for growth on either LA or 4-HV. This supports the hypothesis that they are involved in a reaction after the conversion of LA-CoA to 4HV-CoA. To confirm that the association between LvaA and LvaB is important for enzymatic activity, the following enzymatic combinations were tested: i) LvaA, LvaD and LvaE, ii) LvaB, LvaD, and LvaE, iii) LvaAB, LvaD and LvaE. A decrease of 4HV-CoA and an increase of the predicted 4PV-CoA intermediate was seen only when all four of the enzymes were present. See FIG. 5A. Tandem mass spectrometry was used to verify the identity of 4PV-CoA. See FIGS. 6A, 6B, 6C, and 6D. We compared the MS/MS spectrum of 4HV-CoA (FIG. 6A; assignment of selected fragments shown in FIG. 6B) and the MS/MS spectrum of 4PV-CoA (FIG. 6C; assignment of selected fragments shown in FIG. 6D) and detected major ion fragments at m/z 786.191, 537.106 and 519.095 (4HV-CoA) and 866.158, 617.072 and 599.061 (4PV-CoA). For each compound, these fragments can be assigned to the cleavage of a P—O bond, an O—C bond and the dehydration of O—C cleaved product, respectively (FIGS. 6B and 6D). Both compounds are fragmenting at the same bonds, but the resulting m/z values for the daughter ions differ by 79.967. This mass corresponds to the m/z of PO₃H⁻, supporting the existence of the phosphorylated 4HV-CoA species, 4PV-CoA.

### lvaC

The final step in the hypothesized pathway is the formation of 3HV-CoA. Given that the combination of LvaABDE was responsible for generating 4PV-CoA and no 3HV-CoA was detected in these reactions, it was postulated that LvaC was responsible for the final conversion steps. LvaC has homology to the dehydrogenase family of enzymes and 30% amino acid sequence identity to the E. coli acyl-CoA dehydrogenase protein. The ΔlvaC strain was unable to grow on LA, but grew weakly on 4HV. LvaC was purified as an MBP fusion and the resulting protein pellet displayed a yellow hue. This is often indicative of a co-purified flavin and an absorbance scan of the protein revealed absorbance maxima that are consistent with a flavin co-factor (data not shown). When the LvaC sample was treated with trichloroacetic acid and centrifuged, a white protein pellet and a yellow hued supernatant were observed (data not shown). This indicates that the co-factor was not covalently bound to LvaC. Dijkman, W. P. & Fraaije, M. W. Discovery and characterization of a 5-hydroxymethylfurfural oxidase from Methylovorus sp. strain MP688. Appl. Environ. Microbiol. 80, 1082-1090 (2014).

When LvaC was added to the in vitro reaction mixture, the concentrations of reaction intermediates (LA-CoA, 4HV-CoA, 4PV-CoA) were reduced while the abundance of 3HV-CoA and a pentenoyl-CoA species increased. See FIG. 5A. This species is likely either 2-pentenoyl-CoA and/or 3-pentenoyl-CoA, which could not be resolved with the methods used. Both compounds eluted at the same retention time with the same molecular mass. To test if LvaC is solely responsible for the conversion of 4PV-CoA to 3HV-CoA, a two-step reaction was used. First, we performed the LvaABDE reaction with LA, CoA, ATP, NAD(P)H and separated the CoA products from the enzymes. To the enzyme-free mixture, we added LvaC without additional co-factors. After 30 min, we observed signals for both pentenoyl-CoA and 3HV-CoA. This indicated that the putative oxidoreductase, LvaC, is responsible for both the removal of the phosphate group to produce the enoyl-CoA and the hydration of the enoyl to the 3-hydroxyl compound.

To reconstitute the entire pathway, a time course reaction with all five Lva enzymes and LA as the starting substrate was performed. Over time, a rapid increase in pentenoyl-CoA was observed followed by a slow disappearance that mirrored the increase in the 3HV-CoA signal. See FIG. 5B. This suggests that the hydration reaction may be the limiting step in the overall pathway.

### lvaFG

Based on homology alignments, lvaG is predicted to encode a protein with 95% amino acid sequence identity to a Pseudomonas aeruginosa cation acetate symporter and/val shares 33% amino acid sequence identity with the E. coli inner membrane protein Yhjb (BLAST). Sequence alignments of lvaF orthologs indicate that lvaF and lvaG are found with the same spatial relationship to each other in many organisms (data not shown). These proteins are likely involved in organic acid transport but are unlikely to be involved in the catabolism of LA given that they were not necessary for the enzymatic conversion of LA to 3HV-CoA in vitro.

### Conferring Growth on Levulinic Acid to E. coli LS5218

To demonstrate the ability of the lvaABCDE to enable LA catabolism, we transformed Escherichia coli LS5128 [fadR601, atoC(Con)], a common strain used in studies of organic acid catabolism, with a plasmid linking LvaABCDE expression to an anhydrotetracycline inducible promoter (pJMR5). See Jenkins, L. S. & Nunn, W. D. Genetic and molecular characterization of the genes involved in short-chain fatty acid degradation in Escherichia coli: the ato system. J Bacteriol 169, 42-52 (1987). This strain failed to grow when cultured in minimal LA media. Adaptive evolution of this strain yielded two mutants that grew robustly on media with LA as the sole carbons source. The two mutants had three common mutations 1.) an altered RBS controlling LvaA translation, 2.) a disruption in fadE, and 3.) a disruption in atoC. See Table 8 in the Examples. Freshly created deletion mutants harboring pJMR32 (a variant of pJMR5 with a putative strong RBS) demonstrated that the fadE deletion and the atoC deletion were both beneficial. These deletions likely prevent side reactions catalyzed by FadE and AtoDA (activated by AtoC) that compete with the desired catabolic flux to central metabolism.

Thus described herein is an operon responsible for assimilating LA into the β-oxidation pathway of P. putida. Through an integrated genetic and in vitro biochemistry study, it has been demonstrated herein that the genes lvaABCDE are upregulated in the presence of LA and are sufficient for the conversion of LA to 3HV-CoA, an intermediate of native β-oxidation. Removing any enzyme from the reaction mixture abolished 3HV-CoA production, indicating all five genes are necessary for this pathway. The biochemical assays confirmed the presence of 4PV-CoA, an intermediate previously observed in the metabolism of LA in rat livers. In sum, the pathway consumes at least two (2) ATP and one reducing equivalent to produce 3HV-CoA. See the pathway in FIG. 3. β-oxidation of 3HV-CoA to acetyl-CoA and propionyl-CoA would recover the reducing equivalent. Given the energy demands of the pathway, growth on LA should be performed aerobically or in the presence of an alternative electron acceptor to enable ATP synthesis via respiration.

Like many catabolic pathways, expression of the lva operon is regulated by the presence of the pathway substrates. Using a transcriptional reporter assay, we demonstrated that the lva operon is upregulated by a transcriptional activator encoded by the divergent lvaR gene. Additionally, we suspect that the lva operon is also regulated by Crc, a global carbon catabolite repressor. Crc is an mRNA binding protein that prevents protein translation when bound to a specific mRNA sequence in P. putida, AAnAAnAA. This sequence pattern is found immediately upstream of lvalE (data not shown), which encodes an acyl-CoA synthetase that initiates the pathway. The presence of the Crc target sequence suggests that the operon is also subject to P. putida's carbon catabolite repression system which may explain the diauxic growth curves observed for mixtures of glucose and LA.

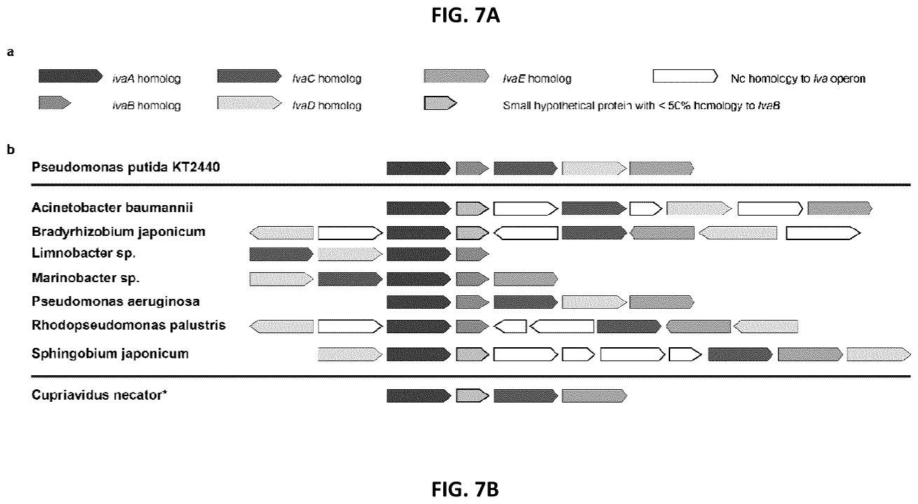

The lva operon is highly conserved among the various Pseudomonas species (data not shown). Gene clusters comprised of the main enzymatic proteins can also be found in a variety of alpha-, beta- and gamma-proteobacteria, graphically represented in FIGS. 7A and 7B. The alpha-proteobacteria species (Azospirillum, Bradyrhizobium, Rhodopseudomonas, Sphingobium) are primarily isolated from soil environments, similar to Pseudomonas putida. The beta-proteobacteria species (Azoarcus, Limnobacter) and the gamma-proteobacteria species (Acinetobacter, Marinobacter) are isolated from both soil and ocean environments. Cupriavidus nector contains a gene cluster comprised of potential LvaACE homologs, and additionally possesses in that same cluster a small hypothetical protein which could be a functional homolog of LvaB.

Interestingly, the isomerization of 4HV-CoA to 3HV-CoA in P. putida proceeds through a phosphorylated intermediate, 4PV-CoA, a compound also observed in a study of LA metabolism in rat livers. Harris et al., supra. This study suggested the 3HV-CoA was generated via a pathway comprised of complex phosphorylated intermediates. We did not detect MS peaks corresponding to any of these compounds in our in vitro reaction mixtures. Without being limited to any underlying mechanism, and based on changes we observed in total ion abundance over time, we propose that 4PV-CoA is dephosphorylated to an enoyl-CoA and subsequently rehydrated to 3HV-CoA. We suspect that the phosphorylation of 4HV-CoA by LvaAB generates a better leaving group and makes the subsequent dehydration more thermodynamically favorable. However, the mechanism for these last steps remains unclear.

The time course measurements that we collected for the full reaction indicate that the formation of the pentenoyl-CoA happens fairly quickly, but the transition from the pentenoyl-CoA to the 3HV-CoA is a much slower reaction. See FIG. 5B. Our tests indicate that LvaC is capable of converting 4PV-CoA to 3HV-CoA, but those reactions still contain a higher abundance of pentenoyl-CoA compared to 3HV-CoA. A more detailed mechanistic study of the final steps may clarify the specific role of lvaC.

Understanding how LA metabolism works is important because LA is a common byproduct of biomass hydrolysis and is often present in the final feedstock. High concentrations of LA in the feedstock can lead to microbial inhibition and represents an underused source of carbon in traditional sugar fermentations. By discovering the catabolic pathway, the present method is useful to engineer microbes capable of detoxifying the media and/or utilizing LA as a source of carbon, thereby maximizing the overall carbon conversion from biomass into high value products. Additionally, identifying the structure of LA metabolism will improve metabolic models and enable pathway design for novel LA-based bioconversions.

### Converting LA to 2-Butanone

Converting LA catalytically to the solvent 2-butanone (methyl ethyl ketone, MEK) has been reported as feasible, but the approach suffers from energy intensive process conditions and low yields. Serrano-Ruiz, J. C., West, R. M. & Dumesic, J. A. Catalytic Conversion of Renewable Biomass Resources to Fuels and Chemicals. Annu. Rev. Chem. Biomol. Eng. 1, 79-100 (2010). 2-butanone has been produced biologically through the dehydration of 2,3 butanediol with a vitamin B₁₂ dependent diol dehydratase. Yoneda, H., Tantillo, D. J. & Atsumi, S. Biological production of 2-butanone in Escherichia coli. ChemSusChem 7, 92-95 (2014). The direct decarboxylation of LA (a five carbon γ-ketoacid) into 2-butanone has been demonstrated using acetoacetate decarboxylase (adc) from Clostridium acetobutylicum as a biocatalyst, but the enzyme is susceptible to substrate inhibition, limiting its overall productivity. Min, K. et al. Conversion of levulinic acid to 2-butanone by acetoacetate decarboxylase from Clostridium acetobutylicum. Appl. Microbiol. Biotechnol. 97, 5627-5634 (2013).

Using the pathway for LA catabolism, lvaABCDE, in P. putida and the expression of short chain fatty degradation pathways, we evolved two strains of E. coli for utilization of LA as a carbon substrate. The evolved strains were derived from E. coli LS5218, which contains specific mutations for overexpression of β-oxidation (fadR601) and short chain fatty acid degradation genes [atoC(Con)]. The draft LS5218 genome assembly is GCA_002007165.1. We sequenced the genome of the mutants and isolated two key functional deletions required for LA growth. Reconstitution of the isolated mutations in wild type LS5218 revealed one, fadE, to be beneficial. Another mutation (in atoC) also conferred a beneficial growth phenotype. Neither mutation is necessary for growth on LA. Using these strains as a basis, we engineered production of butanone by expressing an acetoacetyl-CoA transferase (AtoDA) and an acetoacetate decarboxylase (ADC) in conjunction with LvaABCDE. Our first-generation engineered strains produced 140 mg/L of butanone from LA.

### Evolving E. coli for Growth on LA

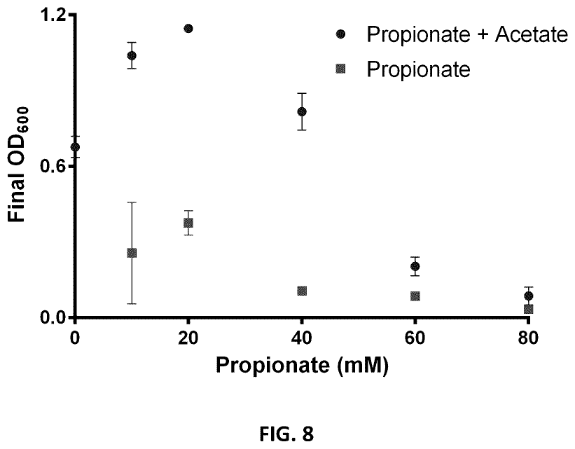

LA is a five carbon acid that degrades into equal moles of acetyl-CoA and propionyl-CoA, and while E. coli contains the necessary genes for propionyl-CoA metabolism, increased propionyl-CoA concentrations are known to be inhibitory. Man, W. J., Li, Y., O'Connor, C. D. & Wilton, D. C. The Binding of Propionyl-Coa and Carboxymethyl-Coa to Escherichia-Coli Citrate Synthase. Biochim. Biophys. Acta 1250, 69-75 (1995). Therefore, we performed a growth study on wild type E. coli MG1655 to evaluate its capability towards propionate catabolism and investigated the growth of E. coli on various concentrations of propionate, with and without acetate as a secondary carbon source. The maximum allowable concentration that stimulated growth was 20 mM propionate, both in the presence and absence of acetate, before growth inhibition was observed. See FIG. 8. Using this information, we designed all LA growth experiments to contain maximum concentration of 20 mM LA to minimize false negative growth phenotypes resulting from propionate toxicity.

Five biosynthetic enzymes are required for catabolizing LA into a common β-oxidation intermediate, encoded by the lva operon from Pseudomonas putida, and lvaABCDE were expressed as an operon in E. coli LS5218 from the plasmid pJMR5. We hypothesized that this combination of expressed enzymes would confer LA catabolism in E. coli, however, initial tests on LA as a sole carbon source did not produce a positive growth phenotype. We then performed a sub-culturing experiment to evolve a strain capable of LA catabolism. The first rounds were conducted with both LA and acetate as available carbon to stimulate growth and allow cells to adapt to the presence of LA. We observed an increase in final cell density with the both carbon sources present compared with the acetate only control, and subsequent culturing was done with LA as the sole carbon source. After 14 rounds of sub-culturing on LA, we isolated two mutant strains, M141 and M142, capable of LA catabolism.

We purified the plasmid, pJMR5, and sequenced it to determine if evolutionary changes were due to plasmid mutations. A mutation in the ribosome binding sequence (RBS) for the lvaABCDE operon was discovered (Table 8), and corresponded to an increase in the predicted strength compared with original sequence. We retransformed the isolated plasmid, designated p2, into wild type LS5218 and the resulting strain did not have the LA growth phenotype, indicating genomic mutations were also necessary (data not shown). To isolate the essential genomic mutations, we submitted strains M141 and M142 for whole genome sequencing after curing out the plasmid. The sequencing results highlighted four mutations in M141 and three mutations in M142 when compared with the genome sequence assembled for wild type E. coli LS5218, with only two common mutations between both strains (Table 8). The common mutations were a point mutation in fadE that resulted in a premature stop codon causing a functional deletion and the insertion of transposons into atoC that also resulted in a premature stop codon and a functional deletion.

### Developing Engineered Strain of E. coli that Catabolizes LA

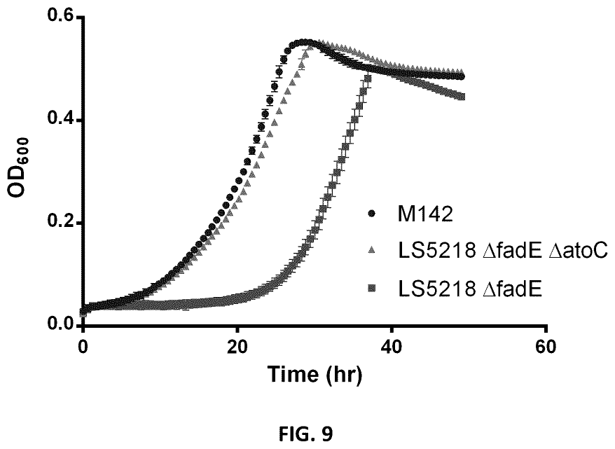

We verified the functional deletion mutations by generating clean knockouts of fadE and atoC as single knockouts and a combined knockout strain using CRISPR-Cas9 mediated genome engineering. We transformed each strain with the plasmid pJMR32, a redesigned pJMR5 with increased RBS strength for LvaA. We examined growth on LA as a sole carbon source for each strain. Wild type LS5218 and LS5218 ΔatoC were unable to grow on LA where as LS5218 ΔfadE and LS5218 ΔatoC ΔfadE grew using LA as the sole carbon source and strain M142 with pJMR32 was used as a positive control. We generated growth curves on LA for strains LS5218 ΔfadE, LS5218 ΔatoC ΔfadE and M142 and found that LS5218 ΔfadE has a significantly longer lag period then LS5218 ΔatoC ΔfadE and M142. See FIG. 9.

In a follow up experiment, the host cell was an E. coli strain in which the lva operon (under aTc induction) and the E. coli fadBA (under IPTG induction) were overexpressed in the host cell. This cell was able to grow on LA without deleting fadE. (Data not shown.) This result indicates that fadBA overexpression is beneficial for E. coli growth on LA. To induce fadBA overexpression in E. coli, fadR is deleted to deregulate those genes (which also deregulates fadE). Therefore, in the preferred host cells, if fadR is deleted, then fadE should also be deleted to minimize competing side reactions. If fadR is not deleted, then overexpression of fadBA is preferred because it maximizes butanone production.

### Establishing Butanone Production

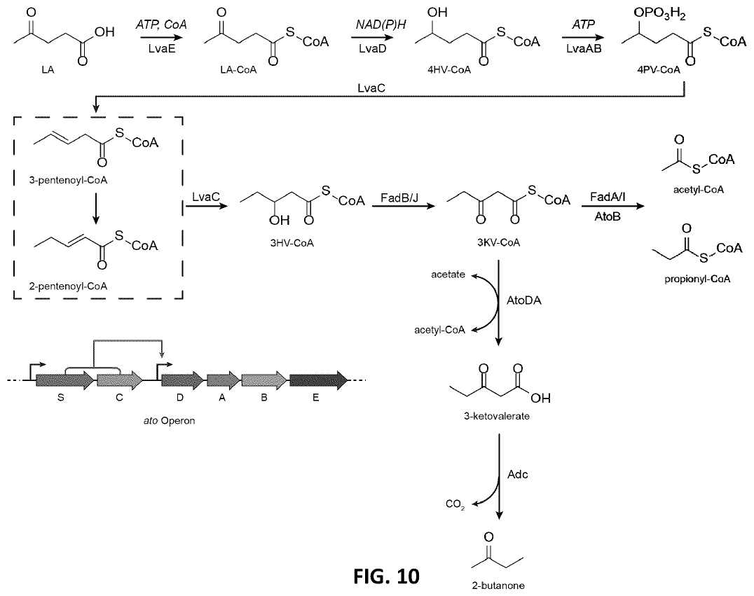

Unlike the previously reported schemes for the production of 2-butanone, we proposed that 2-butanone could be produced through a similar pathway as acetone production. In Clostridium acetobutylicum, acetone is produced through the condensation of two acetyl-CoA molecules to acetoacetyl-CoA (a β-ketoacyl-CoA), which can be liberated to acetoacetate by a CoA transferase and then decarboxylated to acetone by acetoacetate decarboxylase. Our strategy for producing 2-butanone is depicted in FIG. 10. First, LA is catabolized to 3-hydroxyvaleryl-CoA (3HV-CoA) through P. putida enzymes encoded by lvaABCDE. Then a 3-hydroxyacyl-CoA dehydrogenase (encoded by fadB) oxidizes 3HV-CoA to 3-ketovaleryl-CoA (3KV-CoA) followed by the transfer of CoA from 3KV-CoA to acetate to form of 3-ketovalerate through an acetoacetyl-CoA transferase (encoded by atoDA). This conversion can also be accomplished using a short-chain thioesterase or a succinyl-CoA transferase. Finally, acetoacetate decarboxylase (encoded by adc from C. acetobutylicum) converts 3-ketovalerate into butanone and CO₂.

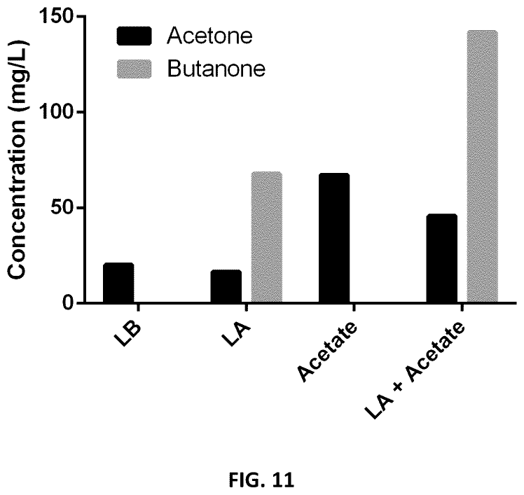

The lvaABCDE and ade genes were co-expressed from the plasmids pJMR32 and pJMR95, respectively. The plasmid pJMR95 is a medium copy plasmid containing the P_{trc} promoter and an origin compatible with pJMR32. Chromosomal overexpression of atoDA genes resulted from the atoC(Con) mutation in host strain E. coli LS5218. To verify the validity of the proposed butanone production pathway, we tested butanone production in the non-optimized strains, eMEK1 (LS5218 ΔfadE) and eMEK12 (LS5218 ΔfadE ΔatoC), containing plasmids pJMR32 and pJMR95. For our experiment, we grew the strains in rich media (LB), added 20 mM of acetate, LA, or both carbons and examined the supernatant for acetone and butanone after 24 hours. We found that eMEK12 was incapable of producing either ketone species, and eMEK1 could produce butanone only in the presence of LA. See FIG. 11. eMEK1 produced up to 140 mg/L of butanone when both LA and acetate were supplied.

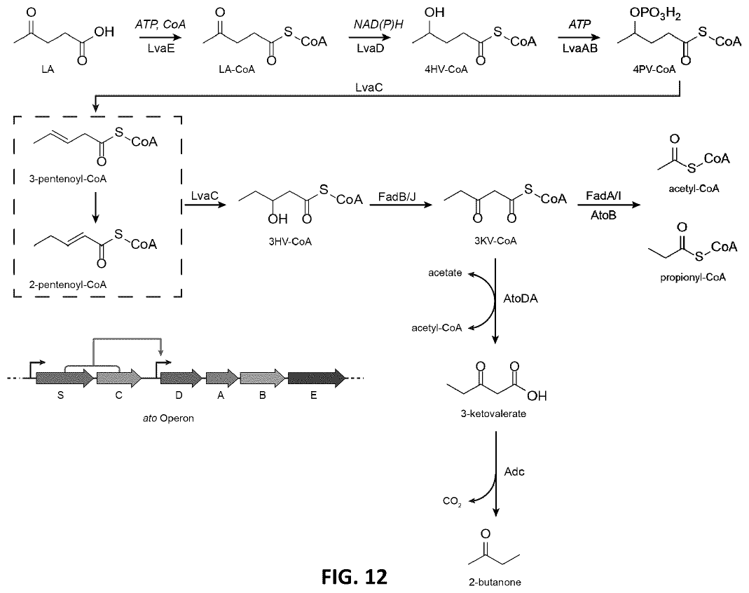

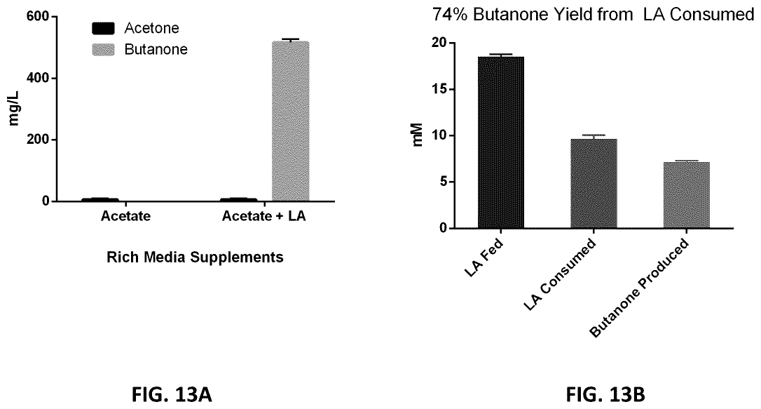

To increase flux of LA towards butanone production and to reduce the formation of acetone, we deleted the competing degradation pathways. We removed the thiolase enzymes encoded by fadA, fadI and atoB to delete the competing degradation pathways for LA catabolism and increase LA flux towards butanone production. See FIG. 12. We tested butanone and acetone production in a more optimized strain, eMEK4 (LS5218 ΔfadEAIJ ΔatoB), using pJMR95 (adc from C. acetobutylicum) and pJMR32-Cm (lvaABCDE with chloramphenicol resistance). This strain did not show an appreciable amount of acetone production and was capable of producing 500 mg/L butanone when both LA and acetate were supplied. See FIGS. 13A and 13B. We also calculated the yield of butanone from LA and determined a 74% measurable yield, indicating we are very close to the theoretical maximum.

### Discussion and Future Directions

The catabolic pathway for LA in P. putida indicated that the last steps in the pathway were undertaken by enzymes involved in β-oxidation. We hypothesized that expression of lvaABCDE in E. coli LS5218, which carries mutations for β-oxidation overexpression, would directly confer E. coli growth on LA. That assumption was proven incorrect. The deletion of fadE is beneficial to allow LS5218 a growth phenotype on LA. FadE is an acyl-CoA dehydrogenase enzyme that catalyzes the formation of the a trans-2-enoyl-CoA from an acyl-CoA compound. Because the LA catabolic pathway terminates at the formation of 3HV-CoA, the final steps to be completed by the E. coli β-oxidation pathway would only involve fadBA, so it remains unclear as to why a fadE is beneficial. We hypothesize that FadE may be active towards LA-CoA, adding a double bond at the 2 position of the γ-ketovaleryl-CoA species and sequestering the molecule from further degradation, however, this is hypothesis is speculative. FadE is an inner membrane protein, thereby complicating efforts to purify an active enzyme and making it hard to ascertain the true nature behind the cellular activity of fadE. Detailed metabolite analysis of LS5218 with lvaABCDE grown in the presence of acetate and LA may reveal insight to the mode of inhibition caused by FadE expression.

The deletion of atoC was not a necessary mutation, but did confer a growth benefit. This mutation was isolated during through the directed evolution process because we were screening for mutants with reduced lag phases, thereby enriching our mutant population with strains containing the early termination sequence. Constitutive activation of the ato regulon by the atoC(Con) mutation in LS5218 causes an overexpression of an acetoacetyl-CoA transferase (encoded by atoDA), an acetyl-CoA acetyltransferase (encoded by atoB) and a short chain fatty acid transporter (encoded by atoE). We propose that the 3-ketovaleryl-CoA intermediate was diverted from the final cleavage step into central metabolites by AtoDA, releasing 3-ketovalerate. The sequestering of LA as 3-ketovalerate reduces overall carbon flow to central metabolites, stunting growth of the E. coli strains until they can adapt for the utilization of 3-ketovalerate. Reducing expression of AtoDA through the deletion of atoC would prevent the formation of the secondary pathway, allowing direct flux of LA to central metabolites. Additionally, AtoE is a short chain fatty acid transporter and overexpression could be causing an increase in the intracellular concentration of LA above a threshold LS5218 is capable of tolerating, causing an extended lag phase. Monitoring intracellular metabolites during the extended lag phase could be useful in isolating the exact cause when compared with the ΔatoC strains.

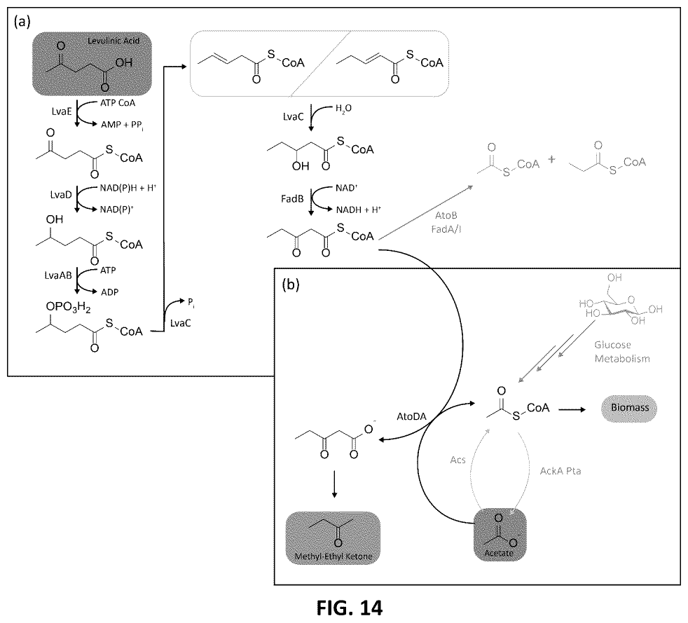

We have demonstrated herein that butanone can be produced from our novel pathway at a final concentration of at least 500 mg/L, which is on par with the previously reported bioconversion processes. Because atoDA encode for a CoA transferase instead of a thioesterase, a short chain acid, such as acetate, is required as a substrate along with 3KV-CoA to produce 3-ketovalerate and acetyl-CoA. In order to optimize the utilization of acetate for the direct formation of 2-butanone, we propose to couple the uptake of acetate with the CoA transferase reaction. Deleting the CoA forming acetyl-CoA synthetase (Acs) and the Pta (phosphotransacetylase) and Ack (acetate kinase) should limit the routes for acetate uptake and couple acetate uptake with 3-ketovalerate production. See FIG. 14.

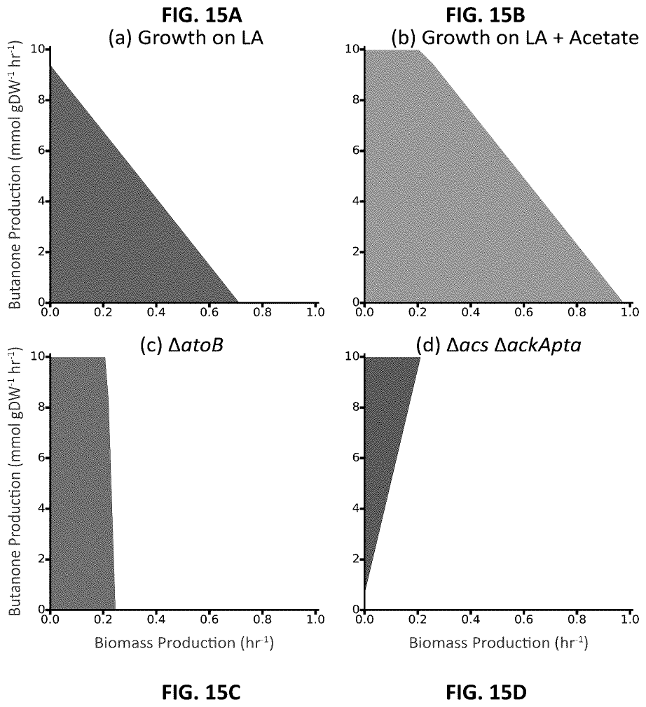

By adding the LA catabolism pathway to the iJO1366 model of E. coli metabolism with a maximum uptake rate of 10 mmol gDW⁻¹ hr⁻¹, we were able to model the growth of E. coli using LA as its sole carbon source with a predicted growth rate of 0.71 hr⁻¹. By adding reactions for the acetoacetyl-CoA transferase (acting on 3KV-CoA) and acetoacetate decarboxylase (acting on 3-ketovalerate), we saw a nearly direct trade-off between 2-butanone production and biomass production, with no butanone production at the limit of maximum growth rate (0.71 hr⁻¹) and no growth at the limit of maximum butanone production (9.4 mmol gDW⁻¹ hr⁻¹). See FIG. 15A. Adding externally supplied acetate increases the maximum predicted butanone production rate to 10 mmol gDW⁻¹ hr⁻¹ (complete conversion) by allowing E. coli to grow on acetate and convert all LA to butanone. See FIG. 15B.

To prevent LA from being used as a carbon source, we knocked-out the reaction corresponding to AtoB, which decreased the maximum predicted growth rate to 0.24 hr⁻¹ while maintaining the maximum predicted 2-butanone production rate to 10 mmol gDW⁻¹ hr⁻¹. See FIG. 15C. It is important to note that, because the iJO1366 model does not include reactions for odd-chain β-oxidation, the in silico deletion of β-oxidation-related reactions was not necessary. We then extended this to a growth-coupled strategy for which E. coli is required to produce butanone to grow. By knocking out acetate fermentation (reactions ACKr and PTAr) and acetyl-CoA synthesis from acetate (reaction ACS), the only way for E. coli to make acetyl-CoA is by transferring a CoA from 3HV-CoA to exogenously supplied acetate. In this case, a maximum predicted 2-butanone production rate of 10 mmol gDW⁻¹ hr⁻¹ is achieved (complete bioconversion) simultaneously with the maximum predicted growth rate of 0.21 hr⁻¹. See FIG. 15D. This demonstrates the possibility of a growth-coupled direct bioconversion of LA to butanone.

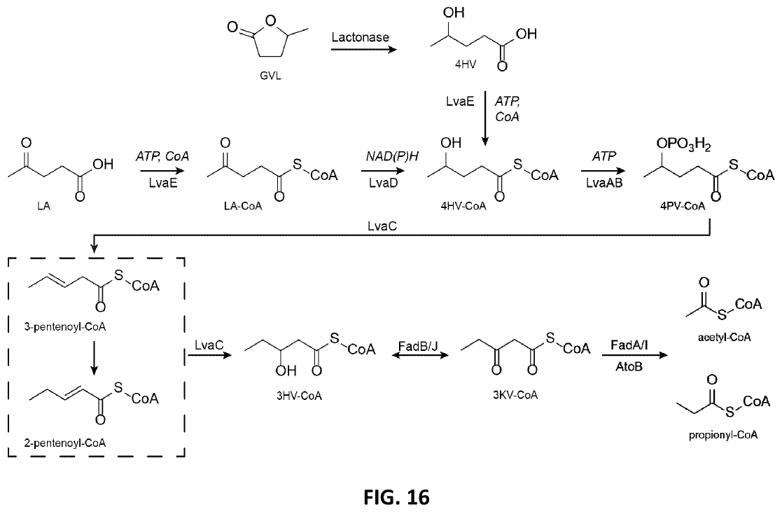

GVL is a derivative of LA that can be produced from a hydrogenation and a dehydration reaction and has been shown to be an effective green solvent in the dissolution of lignocellulosic biomass. See Alonso, D. M., Wettstein, S. G. & Dumesic, J. A. Gamma-valerolactone, a sustainable platform molecule derived from lignocellulosic biomass. Green Chem. 15, 584 (2013); Luterbacher, J. S. et al. Nonenzymatic Sugar Production from Biomass Using Biomass-Derived gamma-Valerolactone. Science (80-.). 343, 277-281 (2014); and Luterbacher, J. S. et al. Lignin monomer production integrated into the γ-valerolactone sugar platform. Energy Environ. Sci. 8, 2657-2663 (2015). As a lactone species, GVL is susceptible to ring opening under basic conditions where it forms 4-hydroxyvalerate, an intermediate in the levulinic acid catabolic pathway. See FIG. 16. Though most bacteria do not thrive under basic pH conditions, some bacteria contain enzymes called lactonases that enzymatically open lactone rings into the corresponding acid. See Ng, F. S. W., Wright, D. M. & Seah, S. Y. K. Characterization of a phosphotriesterase-like lactonase from Sulfolobus solfataricus and its immobilization for disruption of quorum sensing. Appl. Environ. Microbiol. 77, 1181-1186 (2011); Onakunle, O. a., Knowles, C. J. & Bunch, a. W. The formation and substrate specificity of bacterial lactonases capable of enantioselective resolution of racemic lactones. Enzyme Microb. Technol. 21, 245-251 (1997); and Carlier, A., Chevrot, R., Dessaux, Y. & Faure, D. The assimilation of gamma-butyrolactone in Agrobacterium tumefaciens C58 interferes with the accumulation of the N-acyl-homoserine lactone signal. Mol. Plant. Microbe. Interact. 17, 951-7 (2004). We hypothesize that P. putida can be engineered for GVL catabolism if a lactonase with activity towards γ-lactones is heterologously expressed.

Lactones often exist in nature as N-acyl homoserine lactones, a common component of bacterial quorum sensing systems, and as a defense mechanism many bacteria have evolved lactonases to cleave the lactone structure, which can work towards quench the sensing signal. See Uroz, S., Dessaux, Y. & Oger, P. Quorum sensing and quorum quenching: The Yin and Yang of bacterial communication. ChemBioChem 10, 205-216 (2009); Chow, J. Y. et al. Directed evolution of a thermostable quorum-quenching lactonase from the amidohydrolase superfamily. J. Biol. Chem. 285, 40911-20 (2010); and Hiblot, J., Gotthard, G., Chabriere, E. & Elias, M. Structural and enzymatic characterization of the lactonase SisLac from Sulfolobus islandicus. PLoS One 7, e47028 (2012). Due to quorum sensing responses often being linked with pathogenicity, the function of various classes of lactonase have been studied extensively. (Id.) We have compiled a list of five lactonases with reported activity towards γ-lactones, or GVL specifically: Bacillus thuringiensis (protein AiiA), Rhodococcus erythropolis (protein QsdA), Sulfolobus islandicus (protein SisLac), Deinococcus radiodurans (protein DrPLL), and Geobacillus kaustophilus HTA426 (protein GKL).

Preliminary experiments have shown that three of the selected lactonases (DrPLL, GKL, and QsdA) have activity towards degrading GVL and when heterologously expressed in P. putida can confer growth on GVL as a sole carbon source. GVL is capable of supporting P. putida growth, but this growth is severely hindered by an extended lag phase when compared with the related carbon sources of LA and 4HV (data not shown). Directed evolution on GVL media could produce a faster metabolic strain and genomic sequencing could isolate key mutations.

We anticipate that integration of the LA catabolic pathway in a heterologous host can expand the possibilities for biological upgrading of a renewable carbon source. Our proposed approaches presented here represent direct bioconversions, where all LA flux is routed through our production pathways. LA can also be used as an alternative source of intracellular propionyl-CoA, which is a starting molecule for odd chain fatty acids and select secondary metabolites. As a common product produced through the chemical hydrolysis of biomass, catabolizing LA can promote production of a variety of chemicals that normally require an exogenous feedstock and help move us towards environmental sustainability.

### Directed Evolution of E. coli LS5218

See Examples section for cells types and chemicals. E. coli was grown at 37° C., unless otherwise stated. Sub-culturing experiments were done with a volume of 5 ml in glass test tubes (20×150 mm, Fisher Scientific) with 250 rpm agitation in a 126 shaker (New Brunswick Scientific). Starting media contained 20 mM LA and 40 mM acetate or 40 mM acetate only for negative control. Cultures were grown for 72 hours and optical density (OD) measurements taken with a Spectronic 20 (Milton Roy Company), then culture were diluted 1:100 into fresh media. Once the OD in the LA and acetate cultures exceeded the OD of the acetate only cultures, further growth media was 20 mM LA only. These cultures were incubated until turbidity was observed visually, then diluted 1:100 into fresh media. This occurred for a total of 14 dilutions steps in LA media, spanning two weeks.

Plasmids were prepped (QIAprep® Miniprep Kits, Qiagen) and sequenced (Functional Biosciences) to find mutations. Plasmids were cured out of mutate strains through serial culturing in rich media (LB broth) and patch plated on LB and LBₖₐₙ₅₀.

### Genome Engineering with CRISPR-Cas9

CRISPR/Cas recombineering was performed following the outlined protocol in Mark Politz's Thesis (Appendix C). See Politz, M. C. Transcription Activator-Like Effectors as Tools for Prokaryotic Synthetic Biology. (University Of Wisconsin-Madison, 2016). This involves the use of the plasmid pMP11, which contains constitutive expression of S. pyogenes cas9, arabinose-inducible λ Red genes, aTc inducible guide RNA (gRNA) targeted to pBR322 ori, temperature sensitive SC101 ori and Amp^{R}. The plasmid containing the gRNA sequence was designated pgRNA and derived from pgRNA-bacteria. Qi, L. S. et al. Repurposing CRISPR as an RNA-γuided platform for sequence-specific control of gene expression. Cell 152, 1173-1183 (2013). E. coli strains containing pMP11 were at 30° C. (due to the temperature sensitive origin) overnight in LB and diluted into fresh SOB media (Green & Sambrook) with arabinose in the morning. Cultures were grown to an OD of 0.4-0.6 and then cells were made electrocompetent. Competent cells were transformed with the pgRNA plasmid and a linear DNA repair template and selected for on LB containing kanamycin and ampicillin. Following colony PCR, correct strains were cured of the pgRNA plasmid by growing overnight in LB_{Amp} and induced with aTc. pMP11 was cured out of strains by growing overnight at 42° C.

### Butanone Production

Butanone production was tested in strains containing pJMR32 (lvaABCDE) and pJMR95 (adc). Media was comprised of LB broth supplemented with 20 mM LA or 33 mM acetate, accordingly. Production runs were performed for 24 hours with 5 ml in glass test tubes (20×150 mm, Fisher Scientific) with 250 rpm agitation in a 126 shaker (New Brunswick Scientific). Supernatant was filter sterilized and run on Restek Stabilwax-DA column (60 m, 0.53 mm ID) with a GC-FID (Shimadzu). Protocol for GC-FID: 40° C. (hold for 4 min) to 250° C. at 5° C./min, H₂ constant flow, linear velocity 40 cm/sec. Injection and detector temperature was 250° C. Minimal media was prepared according to the batch medium recipe by Korz et al. (Korz et al (1995) “Simple fed-batch technique for high cell density cultivation of Escherichia coli.,” J. Biotechnol. 39, 59-65. Riesenberg et al (1991) “High cell density cultivation of Escherichia coli at controlled specific growth rate,” J. Biotechnol. 20, 17-27. doi:10.1016/0168-1656(91)90032-Q.) Ferric ammonium citrate was substituted for Fe(III) citrate. Hereafter this media is referred to as Riesenberg-Korz (RK) media. Kanamycin was used at final concentration of 50 μg/mL and carbenicillin was used at a final concentration of 100 μg/mL.

Plasmid construction was completed using Phusion High Fidelity DNA Polymerase (NEB) for PCR reactions and Gibson assembly. (Gibson et al. (2009) Enzymatic assembly of DNA molecules up to several hundred kilobases,” Nat. Methods 6, 343-5.) Gibson reaction mixtures (2 μL) were transformed into chemically competent E. coli DH5a and cells were plated on LB media with the appropriate antibiotics. Plasmids were verified by sequencing of the cloning junctions.

### EXAMPLES

### Chemicals, Strains, and Media

All chemicals were obtained from Sigma-Aldrich or Fisher Scientific. Bacterial strains and plasmids used in this study are summarized in Table 3. Strains and plasmids are listed in Table 3. E. coli strains were grown at 37° C. and P. putida strains were grown at 30° C. unless otherwise noted. Kanamycin was used at final concentration of 50 μg/ml. 5-Fluorouracil was used at a final concentration of 20 μg/mL.

4-hydroxyvalerate was made through the saponification of γ-valerolactone (GVL). See Martin, C. H. & Prather, K. L. J. High-titer production of monomeric hydroxyvalerates from levulinic acid in Pseudomonas putida. J. Biotechnol. 139, 61-67 (2009). The pH of 2M GVL was increased to a pH of 12, using 10 M sodium hydroxide (NaOH), and incubated for 1 hour. For use in bacterial growth conditions, 4HV stocks were adjusted to a pH of 8 using 5 M HCl. Plasmid construction was completed using Phusion®-brand High Fidelity DNA Polymerase (New England Biolabs Inc., Ipswich, MA) for the PCR reactions and Gibson assembly. See Gibson, D. G. et al. Enzymatic assembly of DNA molecules up to several hundred kilobases. Nat. Methods 6, 343-5 (2009). P. putida genomic DNA sequences retrieved from NCBI database, with the following designations: PP_2791, lvaA; PP_2792, lvaB; PP_2793, lvaC; PP_2794, lvaD; PP_2795, lvaE; PP_2790, lvaR. 2 μL of the Gibson reaction mixture was transformed into chemically competent E. coli DH5α cells and plated on appropriate media. Minimal media was prepared from the following references: M9 minimal media was made according to Green and Sambrook and MOPS minimal media was made according to Neidhardt et al. Kanamycin was used at final concentration of 50 μg/ml. 5-Fluorouracil was used at a final concentration of 20 μg/mL. See Green, M. R. and Sambrook, J., Molecular Cloning: A Laboratory Manual (Fourth Edition), Cold Spring Harbor Laboratory Press, 2012, ISBN-10: 1936113422 and Neidhardt, F. C., Bloch, P. L. & Smith, D. F. Culture medium for enterobacteria. J. Bacteriol. 119, 736-747 (1974).

TABLE 3
Strains and Plasmid List

| | | |
|---|---|---|
| Strain/Plasmid | Relevant genotype/property | Source or Reference |

Strains
Pseudomonas putida

| | | |
|---|---|---|
| KT2440 | Wild Type | ATCC 47054 |
| KTU | Δupp | Altenbuchner et al., |
| | | Appl. Environ. |
| | | Microbiol. 77, |
| | | 5549-52 (2011) |
| ΔlvaR | Δupp ΔPP_2970 | This work |
| ΔlvaA | Δupp ΔPP_2971 | This work |
| ΔlvaB | Δupp ΔPP_2972 | This work |
| ΔlvaC | Δupp ΔPP_2973 | This work |
| ΔlvaD | ΔPP_2974 | This work |
| ΔlvaE | Δupp ΔPP_2975 | This work |

Escherichia coli

| | | |
|---|---|---|
| CC118λpir | Δ(ara-leu), araD, ΔlacX174, galE, galK, | de Lorenzo et al., |
| | phoA, thi1, rpsE, rpoB, argE (Am), recA1, | BMC Microbiol. 11, |
| | lysogenic λpir | 38 (2011). |
| DH5α | F⁻ Φ80lacZΔM15 Δ(lacZYA-argF) U169 | Invitrogen |
| | recA1 endA1 hsdR17 (rₖ⁻,mₖ⁺) | (Waltham, MA, |
| | phoA supE44 thi-1 gyrA96 relA1 λ⁻ | USA) |
| MG1655 | F⁻ λ⁻ ilvG⁻ rfb-50 rph-1 | Coli Genetic Stock |
| | | Center (“CGSC”), |
| | | Yale University, |
| | | 266 Whitney |
| | | Avenue, New |
| | | Haven, CT, USA |
| LS5218 | F⁺ λ⁺ fadR601 atoC512(Const) | CGSC |
| M141 | LS5218 mutant evolved on LA | This work |
| M142 | LS5218 mutant evolved on LA | This work |
| ΔfadE | LS5218 ΔfadE | This work |
| ΔfadE | LS5218 ΔfadE ΔatoC | This work |

ΔatoC
Plasmids

| | | |
|---|---|---|
| pBAM1 | tnpA, Amp^{R}, Kan^{R}, oriR6K | de Lorenzo et al. |
| pJOE6261.2 | upp (from P. putida) Kan^{R}, ColE1 origin | Altenbuchner et al. |
| pJOE-lvaR | pJOE6261.2 with up- and downstream | This work |
| | regions of lvaR | |
| pJOE-lvaA | pJOE6261.2 with up- and downstream | This work |
| | regions of lvaA | |
| pJOE-lvaB | pJOE6261.2 with up- and downstream | This work |
| | regions of lvaB | |
| pJOE-lvaC | pJOE6261.2 with up- and downstream | This work |
| | regions of lvaC | |
| pJOE-lvaE | pJOE6261.2 with up- and downstream | This work |
| | regions of lvaE | |
| pBAD35 | P_{BAD} promoter, Kan^{R}, pBBR1 origin | Lennen et al., |
| | | Biotechnol. Bioeng. |
| | | 106, 193-202 |
| | | (2010) |
| pBAD-lvaA | pBAD35 carrying lvaA | This work |
| pBAD-lvaB | pBAD35 carrying lvaB | This work |
| pBAD-lvaC | pBAD35 carrying lvaC | This work |
| pBAD-lvaD | pBAD35 carrying lvaD | This work |
| pBAD-lvaE | pBAD35 carrying lvaE | This work |
| pK18mobsacB | sacB, Kan^{R}, pMB1 origin | Schafer et al., Gene |
| | | 145, 69-73 (1994). |
| pK18-lvaD | pK18mobsacB containing up- and | This work |
| | downstream regions of lvaD | |
| pJMR74 | pBAD35 with P_{BAD} promoter and araC | This work |
| | replaced with lvaA promoter and lvaR | |
| | (P. putida) carrying sfGFP | |
| pBbS2k-mCherry | Kan^{R}, SC 101 ori, P_{Tet} promoter, mCherry | Addgene, 75 Sidney |
| | | St. #550A, |
| | | Cambridge, MA, |
| | | USA) |
| pJMR5 | pBbS2k carrying lva operon in front of | This work |
| | mCherry | |
| p2 | pJMR5 mutant evolved on LA | This work |
| pJMR32 | pJMR5 with increased RBS for lva operon, | This work |
| | mCherry removed | |

### Transposon Library and Screening

The transposon library was created following a protocol adapted from Martinez-Garcia et al. Martínez-García, E., Calles, B., Arévalo-Rodríguez, M. & de Lorenzo, V. pBAM1: an all-synthetic genetic tool for analysis and construction of complex bacterial phenotypes. BMC Microbiol. 11, 38 (2011). Suicide vector delivery was achieved through bi-parental mating. Overnights of P. putida KT2440 and E. coli CC118λpir with pBAM1 were grown with appropriate antibiotics. From overnight cultures, 1 mL of cells was pelleted by centrifugation, washed with 10 mM MgSO₄, and resuspended in 1 mL of 10 mM MgSO₄. Cells were mixed in a 1:1 ratio into a final volume of 1 mL 10 mM MgSO₄, with the final concentration of each strain at an OD₆₀₀ of 0.03 (3×10⁷ cells). The mixture was concentrated down to 30 μL and plated on 0.22 μm filter paper. The filter paper was incubated for 16 hrs on LB agar plates at 30° C. After incubation, the filter paper was removed from the plate and transferred into a 1.5 mL microfuge tube with 1 mL of 10 mM MgSO₄. The cells were resuspended through vortexing and plated onto kanamycin selective M9 citrate plates, to isolate P. putida cells with transposon insertions. P. putida transposon library was screened by replica plating colonies from the M9 citrate plates onto LB, M9 glucose and M9 LA plates supplemented with kanamycin. Positive hits were identified as colonies that exhibited growth on LB and glucose plates but not on LA plates.

### RNA Extraction

Wild type P. putida KT2440 cells were grown in MOPS minimal media supplemented with 20 mM LA to OD₆₀₀ 0.8. 10 OD-mL were collected by centrifugation at 5000×g for 10 minutes at 4° C. in Beckman Coulter Allegra X-15R. The supernatant was decanted and the pellet frozen at −80° C. for 24 hrs. The RNA extraction protocol is adapted from Pinto et al. Pinto, F. L., Thapper, A., Sontheim, W. & Lindblad, P. Analysis of current and alternative phenol based RNA extraction methodologies for cyanobacteria. BMC Mol. Biol. 10, 79 (2009). The frozen pellet was thawed, resuspended in 1.5 mL Trizol and transferred to a 2.0 mL microfuge tube. The suspension was incubated for 5 minutes at 95° C. and then for 5 minutes on ice. After the incubation, 300 μL chloroform was added and the tube shaken vigorously for 15 seconds. The Trizol-chloroform mixture was incubated at room temperature for 15 minutes and then centrifuged for 15 minutes at 12000×g and 4° C. The upper phase was transferred to a fresh tube and an equal volume of isopropanol was added. This mixture was incubated for 10 minutes at room temperature and then centrifuged for 10 minutes at 12000×g and 4° C. The supernatant was discarded and the pellet resuspended in 1 mL of 75% ethanol. This was centrifuged for 5 minutes at 8000×g and 4° C. The supernatant was discarded, the pellet air dried for 3 minutes and then resuspended in 100 uL RNase-free water and stored at −80° C.

### Transcription Start Site Isolation

The transcription start site for genes lvaR and lvaA were isolated using an adapted 5′ Race protocol from Schramm et al. Schramm, G., Bruchhaus, I. & Roeder, T. A simple and reliable 5′-RACE approach. Nucleic Acids Res 28, E96 (2000). The RNA isolated from P. putida KT2440 was treated with the TURBO DNA-free™ Kit from Invitrogen, catalog no. AM1907 (a subsidiary of ThermoFisher Scientific, Waltham, MA) to remove any contaminating DNA. The Promega GoScript-brand RT PCR kit was used to generate cDNA using 1 μL of a 10 μM gene specific oligo (JMR2 for lvaR and JMR287 for lvaA) instead of the random oligo mixture. (Promega Corporation, Madison, WI.) Following the inactivation of the reverse transcriptase, the cDNA was purified using Qiagen PCR Purification kit. (Qiagen Inc., Germantown, MD.) Tailing of the cDNA was achieved using the terminal deoxynucleotidyl transferase (TdT) enzyme from ThermoFisher Scientific. The final reaction mixture contained 1× reaction buffer, 1 pmol cDNA fragments, 60 pmol dGTP or dCTP and 30 U TdT. The reaction was incubated at 37° C. for 15 minutes and then quenched by heating to 70° C. for 10 minutes and the tailed cDNA fragments cleaned up using a Qiagen PCR Purification kit. The tailed cDNA was amplified using GoTaq®-brand Green Master Mix (Promega) with an annealing temperature of 55° C. and an extension time of 30 seconds. Primer GG318 was used for dGTP tailing and ALM244 was used for dCTP tailing. The reverse primer for lvaR was JMR150 and for lvaA was JMR296. The resulting PCR product was submitted for sequencing.

### Polycistronic Verification

Using the DNAse treated RNA isolated from LA grown P. putida KT2440, cDNA for the operon was generated with the Promega GoScript-brand RT PCR kit using 1 μL of a 10 μM gene specific oligo (JMR237). The cDNA was then used as the template for PCR reactions using GoTaq Green Master Mix with an annealing temperature of 55° C. and an extension time of 0:30 seconds. Primers used for each gene are given in Table 4.

TABLE 4
Primer List

| | | |
|---|---|---|
| Primer | | |
| Name | Sequence | Function |

5′Race primers

| | | |
|---|---|---|
| JMR2 | AACCTGGACGGTGAAGAGCG | Reverse primer |
| | (SEQ. ID. NO: 11) | for lvaR cDNA |
| JMR287 | GAACGGACAGGAAGCACAG | Reverse primer |
| | (SEQ. ID. NO: 12) | for lvaA cDNA |
| GG318 | GGCCACGCGTCGACTAGTACC | Amplification |
| | CCCCCCCCCCC | primer for dGTP |
| | (SEQ. ID. NO: 13) | tailing reactions |
| ALM244 | GGCCACGCGTCGACTAGTACGGGH | Amplification |
| | HGGGHHGGGHHG | primer for dCTP |
| | (SEQ. ID. NO: 14) | tailing reactions |
| JMR150 | CCAATGCCCGTAGCAGGTCGC | Reverse primer |
| | (SEQ. ID. NO: 15) | for lvaR |
| JMR296 | GAACTCCTGTTCACGGTCAAG | Reverse primer |
| | (SEQ. ID. NO: 16) | for lvaA |

Operon cDNA Reverse Transcription Primer

| | | |
|---|---|---|
| JMR237 | TCAATGATCGACGGCACCG | Reverse primer |
| | (SEQ. ID. NO: 17) | for operon |
| | | cDNA |

Operon-individual genes

| | | |
|---|---|---|
| JMR3 | ACGCTGTGCTTCCTGTCCGTT | lvaA Forward |
| | (SEQ. ID. NO: 18) | |
| JMR325 | GTTCTTCACCGGACAGATGG | lvaA Reverse |
| | (SEQ. ID. NO: 19) | |
| JMR576 | CCCACGAATTGCTCGAGATC | lvaB Forward |
| | (SEQ. ID. NO: 20) | |
| JMR577 | GCAGGTCGGGCAATGTCG | lvaB Reverse |
| | (SEQ. ID. NO: 21) | |
| JMR290 | CATGCCCGTTCGTGCTTC | lvaC Forward |
| | (SEQ. ID. NO: 22) | |
| JMR572 | CAGGTCCATCATGTTGTCGGC | lvaC Reverse |
| | (SEQ. ID. NO: 23) | |
| JMR330 | ACGAGCCGTGAGGACATCT | lvaD Forward |
| | (SEQ. ID. NO: 24) | |
| JMR293 | CGAGCGCAACTTGTCACC | lvaD Reverse |
| | (SEQ. ID. NO: 25) | |
| JMR294 | GCTGGTGTGCATCAACATCC | lvaE Forward |
| | (SEQ. ID. NO: 26) | |
| JMR571 | GCAGTGGAACATCGGCAAGG | lvaE Reverse |
| | (SEQ. ID. NO: 27) | |
| JMR573 | TGTTATACGCGCGTGTTCG | lvaF Forward |
| | (SEQ. ID. NO: 28) | |
| JMR574 | GGTACACGTAGAACGCCGAC | lvaF Reverse |
| | (SEQ. ID. NO: 29) | |
| JMR575 | CATGGTGTTCGTGCTGTTCACC | lvaG Forward |
| | (SEQ. ID. NO: 30) | |
| JMR579 | GCCGAACAGCAACCTGATCA | lvaG Reverse |
| | (SEQ. ID. NO: 31) | |

Operon-individual genes

| | | |
|---|---|---|
| JMR3 | ACGCTGTGCTTCCTGTCCGTT | lvaA Forward |
| | (SEQ. ID. NO: 32) | |
| JMR289 | CAGGTCGGGCAATGTCG | lvaB Reverse |
| | (SEQ. ID. NO: 33) | |
| JMR576 | CCCACGAATTGCTCGAGATC | lvaB Forward |
| | (SEQ. ID. NO: 34) | |
| JMR578 | GAAGCACGAACGGGCATGG | lvaC Reverse |
| | (SEQ. ID. NO: 35) | |
| JMR301 | GCCGACAACATGATGGACCTG | lvaC Forward |
| | (SEQ. ID. NO: 36) | |
| JMR299 | CGTGGTCCCAGGTTTGATCATC | lvaD Reverse |
| | (SEQ. ID. NO: 37) | |
| JMR292 | GCTCGACACCAACCTCAAGG | lvaD Forward |
| | (SEQ. ID. NO: 38) | |
| JMR333 | GCCAAGAACGCTTCGTAGTC | lvaE Reverse |
| | (SEQ. ID. NO: 39) | |
| JMR11 | CAC GGT GCT GGA TAC CGA | lvaE Forward |
| | CA (SEQ. ID. NO: 40) | |
| JMR574 | GGTACACGTAGAACGCCGAC | lvaF Reverse |
| | (SEQ. ID. NO: 41) | |
| JMR573 | TGTTATACGCGCGTGTTCG | lvaF Forward |
| | (SEQ. ID. NO: 42) | |
| JMR579 | GCCGAACAGCAACCTGATCA | lvaG Reverse |
| | (SEQ. ID. NO: 43) | |

### P. putida Knockouts

The genetic knockout of lvaD was performed following the protocol from Schafer et al. Schäfer, A. et al. Small mobilizable multi-purpose cloning vectors derived from the Escherichia coli plasmids pK18 and pK19: selection of defined deletions in the chromosome of Corynebacterium glutamicum. Gene 145, 69-73 (1994). Knockouts of the remaining genes in P. putida were performed following the protocol from Graf et al. Graf, N. & Altenbuchner, J. Development of a method for markerless gene deletion in Pseudomonas putida. Appl. Environ. Microbiol. 77, 5549-52 (2011). Knockout constructs were designed with 500 bp of homology up and down stream of the deletion site. This region was cloned into the pJOE vector backbone. This suicide vector was transformed into P. putida KT2440 Δupp (P. putida KTU) through electroporation and colonies that successfully integrated the plasmid into the chromosome were selected on LBₖₐₙ plates. A colony was then grown in LB media overnight to cure the counter-selection cassette. Various dilutions of the overnight culture were plated on LB_{5-FU} plates to isolate colonies that had successfully excised the plasmid insertion. Colonies were then screened by colony PCR to isolate deletion strains.

### Transcriptional Reporter Assay

P. putida KT2440 was transformed with pJMR74 through electroporation. pJMR74 is a broad host range plasmid containing a kan resistance marker and the predicted regulator for the lva operon, lvaR. Expressed divergent of lvaR is sfGFP cloned under the native promoter for lvaA. P. putida KT2440 containing empty vector pBAD35 was used as the no fluorescence control. Overnights of P. putida+pJMR74 or pBAD35 were inoculated at an OD600 of 0.05 in LB+kan50+20 mM of the appropriate carboxylic acid (acetate, propionate, butyrate, valerate, LA, 4HV, or hexanoate). Final time points were taken at 24 hours in a Tecan infinite m1000, with OD600 absorbance measured at 600 nm and fluorescence measured with an excitation of 485 nm and emission of 510 nm. Standard deviation error propagation was performed for the normalization of fluorescence and optical density measurements.

### Protein Production and Purification

Vectors were constructed using the pET28b backbone and individually cloned genes from the P. putida genome. The plasmid containing lvaAB was constructed using the pET28b backbone and the lvaAB genes cloned as an operon directly out of P. putida's genome. E. coli BL21 (DE3) strains with sequenced verified plasmids were grown at 37° C. in LB. Cultures were induced with 1 mM isopropyl-β-D-thiogalactopyranoside (IPTG) at an OD₆₀₀ of 0.4. The cultures were then chilled on ice for 10 minutes before incubation at 16° C. for 18 hours in New Brunswick Incubator I-26. Then the cultures were centrifuged for 20 minutes at 5000×g in a Beckman Coulter Avanti J-E centrifuge. The supernatant was decanted and the cells resuspended in 30 mL of LB before another centrifugation at 5000×g for 20 minutes. The supernatant was removed and pellets stored at −80° C. for at least 24 hours.

### Purification of His6-(lvaE) and Maltose Binding Protein (MBP)-Tagged Proteins (lvaABCD)

Frozen cell pellets were thawed on ice and resuspended in His6-lysis buffer (50 mM Na₂HPO₄, 300 mM NaCl, 10 mM imidazole, 2 mM DTT, pH 8.0) supplemented with 2 μL of benzonase or MBP-lysis buffer (20 mM Tris-HCl, 200 mM NaCl, 1 mM EDTA, 1 mM DTT, pH 7.4) supplemented with 2 μL of benzonase. Cell suspensions were sonicated 3 times using the program: 1.5 second pulse, 1.5 second pause, 40% duty, for a total of 30 second. Between each sonication cycle, the solution was stored on ice for 5 minutes. Lysed cells were centrifuged at 25,000×g at 4° C. for 30 min and the supernatant filtered through a 0.45 μm filter.

For the purification of His₆-tagged proteins, a GE Äkta Start System with a 1 mL HisTrap HP column and a constant flow rate of 1 mL/minute was used. 5 column volume (CV) of wash buffer (50 mM Na₂HPO₄, 300 mM NaCl, 40 mM imidazole, 2 mM DTT, pH 8.0) was used to equilibrate the column. (GE Healthcare Life Sciences, Pittsburgh, PA.) The sample was loaded and washed with 15 CV wash buffer. The protein was eluted with 5 CV elution buffer (50 mM Na₂HPO₄, 300 mM NaCl, 250 mM imidazole, 2 mM DTT, pH 7.8). 1 mL fractions of eluted protein were collected. A GE PD-10 desalting column was used to buffer exchange the protein into the desalting buffer (100 mM Tris, 4.1 M glycerol and 2 mM DTT). An AmiconR-brand Ultra 4 mL Centrifugal Filter with a 10 kDa cut-off size was used to concentrate the protein. (MilliporeSigma, Billerica, MA.) Each protein was stored at −80° C. until use.

For the purification of MBP-tagged proteins, a GE Äkta Start System with a 1 mL MBPTrap HP column and a constant flow rate of 1 mL/minute was used. 5 column volume (CV) of wash buffer was used to equilibrate the column. The sample was loaded and washed with 15 CV wash buffer. The protein was eluted with 5 CV elution buffer (20 mM Tris-HCl, 200 mM NaCl, 1 mM EDTA, 1 mM DTT, 10 mM maltose, pH 7.4). 1 mL fractions of eluted protein were collected. A GE PD-10 desalting column was used to buffer exchange the protein into the desalting buffer (100 mM Tris, 4.1 M glycerol and 2 mM DTT). An Amicon® Ultra 4 mL Centrifugal Filter with a 10 kDa cut-off size was used to concentrate the protein. The protein was stored at −80° C. until use.

### LvaAB Pulldown Experiment

All proteins for the pulldown experiment were purified on a 1 mL MBPTrap HP column, as previously described, regardless of the protein tag. LvaA was tagged with an N-terminal MBP tag. LvaAB was designed with LvaA tagged with an N-terminal MBP tag and LvaB untagged. Both proteins were expressed from the same construct as they appear in a native operon. For controls, LvaA contained an N-terminal His tag and was expressed with the native LvaB and the last control was an N-terminal MBP tagged LvaA containing a frameshift stop codon expressed with native LvaB. The purified proteins were analyzed on a 15% SDS-page gel to determine the major protein products.

### Enzyme Assays and Metabolite Purification

All in vitro enzyme assays were performed in a 30° C. water bath at a pH of 6.5 and contained 50 mM Tris-HCL, 1 mM MgCl₂, and 2 mM DTT. Final reaction concentrations included the following components, depending on enzymes added: 0.5 mM LA, 0.55 mM CoA, 0.55 mM ATP (1.05 mM ATP when lvaAB were present), 0 mM NAD(P)H (0.55 mM NAD(P)H when lvaD was present). Final protein concentrations were: LvaA (0.2 μM), LvaB (0.8 μM), LvaAB (0.4 μM), LvaC (0.4 μM), LvaD (0.2 μM), and LvaE (0.2 μM) (data not shown). The in vitro enzyme assays were incubated for 30 minutes, excluding the time course which was incubated for various intervals up to 60 minutes. Reaction metabolites were purified following a modified protocol from Zhang, G. F. et al. Catabolism of 4-hydroxyacids and 4-hydroxynonenal via 4-hydroxy-4-phosphoacyl-CoAs. J. Biol. Chem. 284, 33521-33534 (2009). Reactions were quenched by adding methanol/water 1:1 containing 5% acetic acid in a 1:1 volume ratio (extraction buffer). Quenched reactions were run on a 1 mL ion exchange column prepacked with 100 mg 2-2(pyridyl) ethyl silica gel from MilliporeSigma. The column had been preconditioned with 1 mL methanol followed by 1 mL of extraction buffer. Metabolites load on the column were washed with 750 μL extraction buffer before being eluted with 1 mL of 4:1 methanol/250 mM ammonium formate, pH 6.3 and 1 mL methanol. Samples were dried using Thermo Scientific Savant SC250EXP Speedvac Concentrator and stored at −80° C. until LC/MS analysis. Samples for LC/MS analysis were resuspended in 100 uL 50 mM ammonium formate.

### Liquid Chromatography Mass spectrometry (LC/MS, LC/MS/MS)

Samples were analyzed using an HPLC-MS/MS system consisting of a Vanquish™ UHPLC system (Thermo Scientific) coupled by electrospray ionization (ESI; negative polarity) to a hybrid quadrupole-high-resolution mass spectrometer (Q Exactive orbitrap, Thermo Scientific) operated in full scan mode for detection of targeted compounds based on their accurate masses. Properties of Full MS-SIM included resolution of 140,000, AGC target of 1E6, maximum IT of 40 ms, and scan range from 70-1000 m/z. Liquid chromatography (LC) separation was achieved using an ACQUITY UPLC® BEH C18 (2.1×100 mm column, 1.7 μm particle size; Part No. 186002352; Ser. No. 02623521115711; Waters, Milford, MA). Solvent A was 97:3 water:methanol with 10 mM tributylamine (TBA) adjusted to pH 8.1-8.2 with 9 mM acetic acid. Solvent B was 100% methanol. Total run time was 25 min with the following gradient was: 0 min, 5% B; 2.5 min, 5% B; 5 min, 20% B; 7.5 min, 20% B; 13 min, 55% B; 15.5 min, 95% B; 18.5 min, 95% B; 19 min, 5% B; 25 min, 5% B. Flow rate was 200 μL/min. The autosampler and the column temperatures were 4° C. and 25° C., respectively. Fragmentation of CoA, 4HV-CoA, and phosphorylated 4HV-CoA was achieved using parameters indicated in Table 5.

TABLE 5
Other parameters for targeted MS/MS

| | |
|---|---|
| Resolution | 70,000 |
| AGC target | 1E6 |
| Maximum IT | 40 ms |
| Isolation width | 1.4 (m/z) |
| Fixed first mass | 70 m/z |
| (N)CE/stepped (N)CE | 15, 30, 45 |
| Default charge | 1 |
| Polarity | negative |

### Enzymatic “In Gel” Digestion

“In gel” digestion and mass spectrometric analysis was done at the Mass Spectrometry Facility [Biotechnology Center, University of Wisconsin-Madison]. The digestion was performed as outlined on the website: http://www.biotech.wisc.edu/ServicesResearch/MassSpec/ingel.htm. In short, Coomassie Blue R-250 stained gel pieces were de-stained twice for 5 min in MeOH/H₂O/NH₄HCO₃ [50%:50%:100 mM], dehydrated for 5 min in ACN/H₂O/NH₄HCO₃ [50%:50%:25 mM] then once more for 1 min. in 100% ACN, dried in a Speed-Vac for 2 min., reduced in 25 mM DTT [dithiotreitol in 25 mM NH₄HCO₃] for 30 min. at 56° C., alkylated with 55 mM IAA [iodoacetamide in 25 mM NH₄HCO₃] in darkness at room temperature for 30 min., washed twice in H₂O for 30 sec., equilibrated in 25 mM NH₄HCO₃ for 1 min., dehydrated for 5 min. in ACN/H₂O/NH₄HCO₃ [50%:50%:25 mM] then once more for 30 sec in 100% ACN, dried again and rehydrated with 20 μl of trypsin solution [10 ng/μl trypsin Gold (Promega) in 25 mM NH₄HCO₃/0.01% ProteaseMAX w/v (Promega)]. Additional 30 μl of digestion solution [25 mM NH₄HCO₃/0.01% ProteaseMAX w/v] was added to facilitate complete rehydration and excess overlay needed for peptide extraction. The digestion was conducted for 3 hrs at 42° C. Peptides generated from digestion were transferred to a new tube and acidified with 2.5% TFA [trifluoroacetic acid] to 0.3% final. Degraded ProteaseMAX was removed via centrifugation [max speed, 10 minutes] and the peptides solid phase extracted (ZipTip® C18 pipette tips Millipore, Billerica, MA).

### NanoLC-MS/MS

Peptides were analyzed by nanoLC-MS/MS using the Agilent 1100 nanoflow system (Agilent) connected to a new generation hybrid linear ion trap-orbitrap mass spectrometer (LTQ-Orbitrap Elite™, Thermo Fisher Scientific) equipped with an EASY-Spray™ electrospray source. Chromatography of peptides prior to mass spectral analysis was accomplished using capillary emitter column (PepMap® C18, 3 μM, 100 Å, 150×0.075 mm, Thermo Fisher Scientific) onto which 2 μl of extracted peptides was automatically loaded. NanoHPLC system delivered solvents A: 0.1% (v/v) formic acid, and B: 99.9% (v/v) acetonitrile, 0.1% (v/v) formic acid at 0.50 μL/min to load the peptides (over a 30 minute period) and 0.3 μl/min to elute peptides directly into the nano-electrospray with gradual gradient from 3% (v/v) B to 30% (v/v) B over 77 minutes and concluded with 5 minute fast gradient from 30% (v/v) B to 50% (v/v) B at which time a 5 minute flash-out from 50-95% (v/v) B took place. As peptides eluted from the HPLC-column/electrospray source survey MS scans were acquired in the Orbitrap with a resolution of 120,000 followed by MS2 fragmentation of 20 most intense peptides detected in the MS1 scan from 300 to 2000 m/z; redundancy was limited by dynamic exclusion.

### Data Analysis

Raw MS/MS data were converted to mgf file format using MSConvert (ProteoWizard: Open Source Software for Rapid Proteomics Tools Development. Mascot generic format (mgf) is a standard format for MS/MS searches in proteomics and is commonly used for small molecule MS/MS searching. It was developed by Matrix Science Inc., Boston, MA, which also makes and sells software for generating and manipulating mgf files.) Resulting mgf files were used to search against Pseudomonas putida amino acid sequence database containing a list of common contaminants (5,388 total entries) using in-house Mascot search engine 2.2.07 (Matrix Science) with variable methionine oxidation with asparagine and glutamine deamidation plus fixed cysteine carbamidomethylation. Peptide mass tolerance was set at 15 ppm and fragment mass at 0.6 Da.

### Identification of Organisms with Potential Homologous LA Catabolism Pathways

Possible LvaABCD homologs were identified by performing a BLAST search of each protein sequence against the NCBI non-redundant protein sequence database using the BioPython library. Cock, P. J. A. et al. Biopython: freely available Python tools for computational molecular biology and bioinformatics. Bioinformatics 25, 1422-3 (2009). From the search results, the organism name was extracted from the sequence title and added to a set for each protein. The list of organisms containing the full set of LvaABCD enzymes was found by determining the intersection of the four sets of organism names from the BLAST results from each protein. A similar list was found for those organisms containing only LvaACD homologs. These lists were then used to query the original search results and find the lists of proteins that have homology to the proteins in the Lva pathway

### RB-TnSeq

To further investigate genes involved in LA metabolism, random bar code transposon-site sequencing (RB-TnSeq) was performed for the growth of Pseudomonas Putida on LA and 4HV. RB-TnSeq is an efficient method for determining gene importance under different conditions with high genomic coverage. Wetmore, K. M. M. et al. Rapid Quantification of Mutant Fitness in Diverse Bacteria by Sequencing Randomly Bar-Coded Transposons. MBio 6, 1-15 (2015). A mixture of P1 oligos with variable length N space regions (2-5 nt) was used to “phase” the BarSeq PCR products for sequencing on an Illumina HiSeq4000. See Table 6. A summary of genes identified as interesting is shown in Table 7 including fitness scores for growth on minimal media with LA or 4HV relative to minimal media with glucose or the initial inoculum grown in LB.

TABLE 6
Modified Oligonucleotides used for BarSeq

| Oligo Name | Sequence |
|---|---|
| Barseq_P1 | AATGATACGGCGACCACCGAGATCTACA |
| | CTCTTTCCCTACACGACGCTCTTCCGAT |
| | CTNNNNNGTCGACCTGCAGCGTACG |
| | (SEQ. ID. NO: 44) |
| Barseq_P1_4N | AATGATACGGCGACCACCGAGATCTACA |
| | CTCTTTCCCTACACGACGCTCTTCCGAT |
| | CTNNNNGTCGACCTGCAGCGTACG |
| | (SEQ. ID. NO: 45) |
| Barseq_P1_3N | AATGATACGGCGACCACCGAGATCTACA |
| | CTCTTTCCCTACACGACGCTCTTCCGAT |
| | CTNNNGTCGACCTGCAGCGTACG |
| | (SEQ. ID. NO: 46) |
| Barseq_P1_2N | AATGATACGGCGACCACCGAGATCTACA |
| | CTCTTTCCCTACACGACGCTCTTCCGAT |
| | CTNNGTCGACCTGCAGCGTACG |
| | (SEQ. ID. NO: 47) |

TABLE 7
Genes Identified as Interesting
Genes Identified as Interesting

| Locus | Name | Annotation | LA/Gluc | 4HV/Gluc |
|---|---|---|---|---|
| PP_0364 | bioH | pimeloyl-ACP methyl ester esterase | 0.3 | 0.02 |
| PP_0988 | gcvP-1 | glycine dehydrogenase | −0.02 | −0.003 |
| PP_2332 | — | ATP-dependent zinc protease family | −0.1 | 0.2 |
| PP_2336 | acnA-II | aconitate hydratase | −4.5 | −3.5 |
| PP_2337 | prpF | aconitate isomerase | −4.4 | −3.8 |
| PP_2790 | lvaR | Sigma-54 dependent sensory box protein | −3.9 | −5.0 |
| PP_2791 | lvaA | Aminoglycoside phosphotransferase | −5.2 | −4.2 |
| PP_2792 | lvaB | Hypothetical protein | NA | NA |
| PP_2793 | lvaC | acyl-CoA dehydrogenase/reductase family | −5.2 | −4.1 |
| PP_2794 | lvaD | Oxidoreductase, short chain dehydrogenase/reductase family | −6.5 | −5.3 |
| PP_2795 | lvaE | Acyl-CoA synthetase | 0.4 | −4.6 |
| PP_2796 | lvaF | conserved protein of unknown function | 0.2 | 0.7 |
| PP_2797 | lvaG | acetate permease | 0.1 | 1.7 |
| PP_3741 | mrdA-I | transpeptidase | 0.0 | −0.06 |
| PP_4217 | fpvA | TonB-dependent outer membrane ferripyoverdine receptor | 0.3 | 0.02 |

Important for Fitness in LA and 4HV

| | | | |
|---|---|---|---|
| PP_2217 | enoyl-CoA hydratase | −2.0 | −2.2 |
| PP_2334 | 2-methylisocitrate lyase | −4.9 | −3.3 |
| PP_2335 | methylcitrate synthase | −5.1 | −4.7 |
| PP_3286 | DNA-binding transcriptional repressor PaaX(phenylacetyl-CoA) | −4.3 | −4.1 |
| PP_3753 | Transcriptional regulator, AraC family | −4.8 | −2.6 |
| PP_3754 | Beta-ketothiolase BktB | −5.8 | −3.2 |
| PP_3755 | 3 -hydroxybutyryl-CoA dehydrogenase | −2.9 | −3.1 |

Important for Fitness in LA but not 4HV

| | | | |
|---|---|---|---|
| PP_1291 | PhoH family protein | −2.5 | 0.3 |
| PP_2333 | GntR family transcriptional regulator | −4.5 | −0.7 |
| PP_3121 | transcriptional regulator, LysR family | −4.1 | −0.3 |
| PP_3122 | acetoacetyl CoA-transferase (subunit A) | −2.3 | −0.1 |
| PP_3123 | acetoacetyl CoA-transferase (subunit B) | −3.1 | −0.02 |
| PP_3925 | conserved protein of unknown function | −2.1 | −0.9 |
| PP_4515 | Transcriptional regulator, MarR family | −2.2 | 0.03 |
| PP_4628 | conserved protein of unknown function | −3.6 | −1.3 |

Important for Fitness in 4HV but not LA

| | | | |
|---|---|---|---|
| PP_0951 | Ribosome hibernation promoting factor | 0.2 | −2.4 |
| PP_0995 | Putative sigma factor regulator | −0.5 | −2.6 |
| PP_1328 | Protein MraZ | −0.5 | −4.2 |
| PP_1764 | Phosphoglycolate phosphatase 2 | −1.3 | −2.7 |
| PP_1778 | Lipopolysaccharide ABC export system, permease protein | 0.2 | −4.8 |
| PP_1779 | Lipopolysaccharide ABC export system, ATP-binding protein | 0.003 | −4.0 |
| PP_1968 | TetR family transcriptional regulator | −0.8 | −2.1 |
| PP_2082 | phosphoenolpyruvate synthetase | −0.2 | −2.7 |
| PP_2436 | Transcriptional regulator, LysR family | −0.3 | −2.4 |
| PP_4342 | flagellar synthesis regulator, putative ATPase | −1.4 | −2.0 |
| PP_4571 | cysteine synthase A | −0.1 | −3.5 |
| PP_4762 | Acyl-CoA thioesterase II | 0.3 | −4.3 |

### Methods

### P. putida Library Preparation

We generated a DNA-barcoded transposon mutant library of P. putida KT2440 using previously described methods and resources. (Wetmore et al., supra.) Briefly, we conjugated wild-type P. putida KT2440 with an E. coli strain (WM3064) carrying the transposon vector library pKMW3. pKMW3 is a mariner class transposon vector library containing a kanamycin resistance marker and millions of random 20mer DNA barcodes. Conjugations were performed at 1:1 donor:recipient ratio on LB+diaminopimelic acid (DAP) plates for 6 hours and finally plated on LB plates supplemented with 100 μg/mL kanamycin. The E. coli conjugation strain WM3064 is auxotrophic for DAP and does not grow on media that is not supplemented with this compound. We combined thousands of kanamycin-resistant P. putida colonies into a single tube, made multiple aliquots, and stored these samples at −80° C. for future use. We also extracted genomic DNA and mapped the transposon insertion locations and their associated DNA barcodes via a TnSeq-like Illumina sequencing protocol, as previously described by Wetmore et al. (supra). We named the final, sequenced mapped transposon mutant library Putida_ML5.

### LA and 4HV Growth Experiments

An aliquot of the P. Putida RB-TnSeq library (Putida_ML5) was grown for 5 hours in a shake flask containing 25 mL of LB media with 50 ug/mL Kanamycin Sulfate to late log phase (30° C., 250 RPM). 1 OD₆₀₀*mL of cells were pelleted, decanted, and frozen at −20° C. for barcode sequencing as the time zero inoculum control. 1 OD₆₀₀*mL of cells per treatment were washed with three volumes of minimal media with no carbon source and then resuspended in 2× minimal media with no carbon source for a new OD₆₀₀ measurement. These cells were diluted into 2× minimal media to an OD₆₀₀ of 0.04. This culture was then diluted in half with 2× solutions of each carbon source of interest to a final volume of 10 mL in a culture tube for 4HV and 1.2 mL total volume in the well of a 24-well microplate for LA. The carbon sources tested were 40 mM 4HV (pH adjusted to 7 with NaOH), 40 mM LA (pH adjusted to 7 with NaOH), 20 mM potassium acetate, and 40 mM glucose, each with two replicates. The 4HV and acetate experiments were performed one day and the LA experiments were performed on a different day, each day with its own 40 mM Glucose control. The culture tubes were placed in a shaker incubator (30° C., 250 RPM) until they achieved and OD₆₀₀ of ˜3 for 40 mM Glucose (˜20 hours), ˜0.25 for 20 mM potassium acetate (˜44 hours), or ˜0.3-0.5 for 40 mM 4HV (˜68 hours). For LA, the samples were grown in a 24-well microplate in a Multitron shaker set to 30° C. and 700 rpm. We monitored the OD of the microplate in a Tecan M1000 microplate reader. A 1 mL sample from each culture tube was pelleted and frozen at −20° C. for barcode sequencing.

### BarSeq

A DNA barcode sequencing (BarSeq) was performed as described in Wetmore et al. (supra), with a slight variation in the common P1 oligo design. In this study, a mixture of P1 oligos with variable length N space regions (2-5 nt) was used to “phase” the BarSeq PCR products for sequencing on the Illumina HiSeq4000.

### Data Analysis

Both the TnSeq data and the BarSeq data were processed using analysis scripts as described in Wetmore et al. (supra). Briefly, the fitness of a strain in the normalized log₂ ratio of barcode reads in the experimental sample to barcode reads in the time zero sample. The fitness of a gene is the weighted average of the strain fitness for insertions in the central 10-90% of the gene. The gene fitness values are normalized so that the typical gene has a fitness of zero. The primary statistic t-value is of the form of fitness divided by the estimated variance across different mutants of the same gene. All experiments described herein pass the quality metrics described in Wetmore et al. unless noted otherwise.

### Identifying Genes of Interest

The fitness values reported in Table 7 are the average of 2 replicates. Fitness scores for LA and 4HV relative to glucose were calculated using the following equation:

$$Fitness ( \frac{LA}{Glucose} ) = Fitness ( LA ) - Fitness ( Glucose )$$

Annotations in Table 7 and discussed in the text below were adapted from Dehal, P. S. et al. MicrobesOnline: An integrated portal for comparative and functional genomics. Nucleic Acids Res. 38, 396-400 (2009).

### RB-TnSeq Results are Consistent with the Other Evidence

All genes mentioned above are shown with their fitness scores for growth on LA and 4HV in Table 6. Genes that were identified as transposon library hits have their gene loci highlighted in red italics.

RB-TnSeq analysis suggests the genes identified as constituting the LA metabolism operon lvaABCDEFG as well as the proposed regulator lvaR were important for growth on both LA and 4HV with a few exceptions described as follows. lvaB was excluded from the data summary for growth on LA and 4HV due to insufficient barcode insertions in this small gene and lvaE (shown to not be essential for growth on LA in the main text) shows no phenotype on LA.

RB-TnSeq analysis suggests lvaF and lvaG are not important for growth on LA or 4HV, suggesting they are not required for transport of these metabolites at the concentrations used in the experiments. The positive fitness scores of these genes for growth on 4HV suggest that the 4HV concentrations used in this experiment had negative effects on fitness, an effect that would be alleviated by elimination of import system (See section below: Potential Induction of Quorum-Sensing Systems by γ-Valerolactone). None of the remaining transposon library hits noted herein exhibited interesting phenotypes in the RB-TnSeq experiment, suggesting they may have been dependent upon the transposon library experiment.

In addition to genes identified in above, genes of interest shown in Table 6 were identified using the following criteria:
Important for Fitness in LA and 4HV: Fitness scores lower than −2 for both LA and 4HV.
Important for Fitness in LA but not 4HV: Fitness score for LA lower than −2 and fitness score for 4HV greater than −2.
Important for Fitness in 4HV but not LA: Fitness score for 4HV lower than −2 and fitness score for LA greater than −2.
Enhanced Fitness in 4HV: Fitness score greater than 2 for 4HV.

This list of genes of interest was further refined by eliminating genes that shared a phenotype with growth on acetate as these results were considered not relevant to the scope of this work.

### β-Oxidation of 3-Hydroxyvaleryl-CoA to Propionyl-CoA and Acetyl-CoA by Genes Important for Growth on LA and 4HV

As proposed above, the 3-hydroxyvaleryl-CoA metabolite produced in LA metabolism could be utilized through β-Oxidation to form Propionyl-CoA and Acetyl-CoA. RB-TnSeq analysis helped to identify potential candidate genes for this pathway:
PP_3755 is annotated as a 3-hydroxybutyryl-CoA dehydrogenase, suggesting that this enzyme catalyzes the conversion of 3-hydroxyvaleryl-CoA to 3-ketovaleryl-CoA.
PP_3754 is annotated as a β-ketothiolase, suggesting that this enzyme catalyzes the conversion of 3-ketovaleryl-CoA to propionyl-CoA and Acetyl-CoA.
PP_3753 is annotated as a transcriptional regulator and its location directly upstream of the two previous genes suggests a role in the regulation of these two β-oxidation genes.

### Propionyl-CoA Metabolism by Genes Important for Growth on LA and 4HV

After propionyl-CoA is formed through the mechanism proposed in the previous section, it could be further metabolized to form succinate and pyruvate through the 2-methylcitrate cycle. PP_2337 is annotated as a methylaconitate isomerase (prpF), suggesting that the pathway utilized is the 2-methylcitrate cycle II that passes through a trans-2-methyl-aconitate intermediate. RB-TnSeq analysis helped to identify potential candidate genes for this pathway:
PP_2335 is annotated as a methylcitrate synthase, suggesting that this enzyme catalyzes the reaction of propionyl-CoA with oxaloacetate to form 2-methylcitrate.
PP_2336 is annotated as an aconitate hydratase. PP_2339, an additional gene in close chromosomal proximity but with insufficient BarSeq data for analysis is also annotated as an aconitate hydratase. These results suggest that some combination of these two enzymes catalyze both the conversion of 2-methylcitrate to trans-2-methylaconitate and the downstream conversion of cis-2-methylaconitate to 2-methylisocitrate.
PP_2337 is annotated as a methylaconitate isomerase, suggesting that this enzyme catalyzes the conversion of trans-2-methylaconitate to cis-2-methylaconitate.
PP_2334 is annotated as a 2-methylisocitrate lyase, suggesting that this enzyme catalyzes the conversion of 2-methylisocitrate succinate and pyruvate.
PP_2333 is annotated as a transcriptional regulator and its location directly upstream of the PP_2334-2339 genes suggests a role in the regulation of these propionyl-CoA metabolism genes.

### Potential LA CoA Transferase

lvaE was shown to catalyze the conversion of LA to levulinyl-CoA as well as the conversion of 4HV to 4-hydroxyvalerly-CoA. lvaE is essential for growth on 4HV but not essential for growth on LA, suggesting that there is another enzyme capable of catalyzing the conversion of LA to levulinyl-CoA. PP_3122 and PP_3123 are annotated as acetoacetyl CoA-transferase subunits A and B respectively and are both important for growth on LA but not 4HV, suggesting they could fill the role of the additional catalyst for levulinyl-CoA formation. PP_3121 is also important for growth on LA but not 4HV and is annotated as a transcriptional regulator. Its genomic context suggests it regulates the expression of PP_3122 and PP_3123. This set of genes is analogous to the dhcAB operon involved in catabolism of carnitine in Pseudomonas aeruginosa. PP_3121 shares 72% sequence identity across 95% of its sequence with dhcR (PA1998) and PP_3122 and PP_3123 share 86% and 90% identity across their entire sequences with dhcA (PA1999) and dhcB (PA2000), respectively. dhcR regulates expression of the dhcAB operon encoding a predicted 3-ketoacid CoA-transferase with evidence of activity on 3-dehydrocarnitine. Wargo, M. J. & Hogan, D. A. Identification of genes required for Pseudomonas aeruginosa carnitine catabolism. Microbiology 155, 2411-2419 (2009). PP_3121-PP_3123 could serve a similar role in catabolism of LA.

### Transcriptional Regulators Control both Beneficial and Detrimental Systems for Fitness under LA and 4HV Metabolism

PP_3286 and PP_3753 are annotated as transcriptional regulators and RB-TnSeq analysis suggests they are important for growth on LA and 4HV. The annotation for PP_3286 suggests involvement in the regulation of phenylacetic acid metabolism. As previously stated, genomic context suggests the involvement of PP_3753 in the regulation of the probable β-oxidation genes PP_3754-3755.

PP_3121 and PP_4515 are annotated as transcriptional regulators and RB-TnSeq analysis suggests they are important for growth on LA but not important for growth on 4HV. As previously stated, genomic context suggests PP_3121 regulates expression of the potential acetoacetyl-CoA transferase subunits PP_3122-3123. The regulatory role of PP_4515 is unclear.

Conversely, PP_0995, PP_1328, PP_1968, PP_2333, and PP_2436 are annotated as transcriptional regulators and RB-TnSeq analysis suggests they are important for growth on 4HV, but not important for growth on LA. PP_0995 shares 41% homology across its entire sequence with a gene in Caulobacter crescentus (CC3252) thought to be involved in sigma factor regulation for heavy metal stress, although its regulatory role in Pseudomonas putida is unclear. Kohler, C., Lourenço, R. F., Avelar, G. M. & Gomes, S. L. Extracytoplasmic function (ECF) sigma factor σF is involved in Caulobacter crescentus response to heavy metal stress. BMC Microbiol. 12, 210 (2012). As previously stated, genomic context suggests the involvement of PP_2333 in the regulation of the probable propionyl-CoA metabolism genes PP_2333-2339. The regulatory functions of PP_1328, PP_1968, and PP_2436 are unclear.

PP_0191, PP_1236, PP_2144, PP_3603, and PP_4734 are annotated as transcriptional regulators and RB-TnSeq analysis suggests their deletions are beneficial for growth on 4HV. PP_0191 is annotated as a regulator of alginate bioaccumulation, suggesting a role in biofilm formation. PP_1236 is annotated as a regulator of a glycine cleavage system and a close homolog in Pseudomonas aeruginosa (PA1009) is involved in the regulation of host colonization. Koh, A. Y. et al. Utility of in vivo transcription profiling for identifying Pseudomonas aeruginosa genes needed for gastrointestinal colonization and dissemination. PLoS One 5, 1-14 (2010). PP_2144 has a close homolog in Pseudomonas syringae (psrA) that is involved in the regulation of epiphytic fitness, quorum-sensing, and plant host interactions. Chatterjee, A., Cui, Y., Hasegawa, H. & Chatterjee, A. K. PsrA, the Pseudomonas sigma regulator, controls regulators of epiphytic fitness, quorum-sensing signals, and plant interactions in Pseudomonas syringae pv. tomato strain DC3000. Appl. Environ. Microbiol. 73, 3684-3694 (2007).

PP_3603 and PP_4734 are annotated as fatty acid responsive transcriptional regulators with unknown regulatory roles.

### Potential Induction of Quorum-Sensing Systems by γ-Valerolactone

4HV used in the RB-TnSeq experiments was synthesized from γ-valerolactone as described in the methods section of the main text. As a result, residual γ-valerolactone was likely present in the experiments for growth on 4HV. Several molecules in the lactone family are known to be used as quorum sensing signals in Pseudomonads. Pearson, J. P., Passador, L., Iglewski, B. H. & Greenberg, E. P. A second N-acylhomoserine lactone signal produced by Pseudomonas aeruginosa. Proc. Natl. Acad. Sci. U. S. A. 92, 1490-1494 (1995). Quorum sensing responses would likely cause physiological responses towards the formation of a biofilm in the culture vessel. Cells with disruptions in these regulatory systems would replicate themselves to a higher degree resulting in a perceived increase in fitness as is the case with the transcriptional regulators PP_0191, PP_1236, and PP_2144 discussed above. As γ-Valerolactone is being investigated as a promising solvent for nonenzymatic sugar production from biomass (see Luterbacher, J. S. et al. Nonenzymatic Sugar Production from Biomass Using Biomass-Derived gamma-Valerolactone. Science (80-.). 343, 277-281 (2014)), its effect on the quorum sensing systems of potential platform host organisms for bioprocessing should be further investigated.

### Conferring Growth on Levulinic Acid to E. coli LS5218

E. coli strain LS5218 is commonly studied for the production of polyhydroxyalkanoates (PHAs) and carries two known mutations: a mutation in fadR, which deregulates the genes encoding the β-oxidation enzymes and allows for constitutive expression of the fad genes, and an atoC(Con) mutation that causes constitutive upregulation of the ato operon, an operon responsible for the metabolism of short-chain fatty acids. The mutations in E. coli LS5218 allow for increased uptake and utilization of a wider array of fatty acid chain lengths, and make it especially adapted for the engineering of short chain length-co-medium chain length (SCL-co-MCL) copolymers and poly(3-hydroxybutyrate-co-3-hydroxyvalerate) [P(3HB-co-3HV)].

LA is catabolized into equal moles of acetyl-CoA and propionyl-CoA. While E. coli contains the necessary genes for propionyl-CoA metabolism, elevated propionyl-CoA concentrations are known to be inhibitory. Therefore, we performed a growth study to evaluate growth of E. coli on various concentrations of propionate, with and without acetate as a secondary carbon source. The maximum allowable concentration that stimulated growth was 20 mM propionate, both in the presence and absence of acetate (data not shown). Using this information, we designed all LA growth experiments to contain the maximum concentration of 20 mM LA to minimize false negative growth phenotypes resulting from propionate toxicity.

The five biosynthetic enzymes required for catabolizing LA into a common β-oxidation intermediate (lvaABCDE) were expressed as an operon in E. coli LS5218 from the plasmid pJMR5 (SC101 origin, Pₜₑₜ, kanR). We hypothesized that this combination of expressed enzymes would confer LA catabolism in E. coli. However, in initial trials E. coli LS5128 pJMR5 failed to grow on LA as a sole carbon source (data not shown). Therefore, we performed adaptive evolution, sub-culturing cells into fresh media daily, to evolve a strain capable of LA catabolism. The first three rounds were conducted with both LA and acetate as available carbon to stimulate growth and allow cells to adapt to the presence of LA. In these experiments, we observed an increase in final cell density when both carbon sources were present relative to cultures that were fed only acetate. Subsequent rounds of evolution were conducted with LA as the sole carbon source. After 14 rounds of sub-culturing on LA, we isolated two mutant strains, M141 and M142, capable of robust LA catabolism.

We purified the lvaABCDE expression plasmid from the mutant strains and sequenced it to determine if evolutionary changes were due to plasmid-borne mutations. A mutation in the ribosome binding sequence (RBS) for the lvaABCDE operon was discovered. See Table 8. RBS Calculators predicted an increased translation initiation rate relative to the original sequence. We retransformed the isolated plasmid, designated p2, back into wild type LS5218 and the resulting strain did not have the LA growth phenotype, indicating genomic mutations were also necessary (data not shown). To isolate the essential genomic mutations, we submitted strains M141 and M142 for whole genome sequencing after curing out the plasmid. The sequencing results highlighted five mutations in M141 and four mutations in M142 when compared with the genome sequence assembled for wild type E. coli LS5218 (GCA_002007165.1), with only two common mutations between both strains (Table 8). The common mutations were a point mutation in fadE that resulted in a premature stop codon causing a functional deletion and the insertion of transposons into atoC that also resulted in a premature stop codon and a functional deletion.

TABLE 8
List of mutations from evolved strains M141 and Ml42

| | | | |
|---|---|---|---|
| Position | Gene | Mutation | Change |

Genomic mutations
Common

| | | | |
|---|---|---|---|
| 243014 | fadE | C →T | Trp → stop codon |
| 2323064 | atoC (M141) | Transposable | Early stop codon |
| | | element insertion | |
| 2322858 | atoC (Ml42) | Transposable | Early stop codon |
| | | element insertion | |

M141

| | | | |
|---|---|---|---|
| 205559 | dnaE | G →A | Arg →His |
| 261153 | proB | C →T | His →Tyr |
| 3390059 | aaeR | A →C | Lys →Asn |

M142

| | | | |
|---|---|---|---|
| 2395921 | nuoI | C →T | Ser →Asn |
| 4161154 | fabR | A →C | Thr →Pro |

Plasmid mutations

| | | | |
|---|---|---|---|
| pJMR5 | RBS | G →T | Increased RBS |
| | | | strength |

To verify the importance of the common mutations we generated clean knockouts of fadE and/or atoC using CRISPR-Cas9 mediated genome engineering. Jiang, W., Bikard, D., Cox, D., Zhang, F. & Marraffini, L. a. RNA-guided editing of bacterial genomes using CRISPR-Cas systems. Nat Biotechnol 31, 233-239 (2013). Jiang, Y. et al. Multigene editing in the Escherichia coli genome via the CRISPR-Cas9 system. Appl. Environ. Microbiol. 81, 2506-2514 (2015). Li, Y. et al. Metabolic engineering of Escherichia coli using CRISPR-Cas9 meditated genome editing. Metab. Eng. 31, 13-21 (2015). We transformed each strain with the plasmid pJMR32, a redesigned pJMR5 with predicted increased RBS strength for LvaA. We examined growth on LA as a sole carbon source for each strain. Wild type LS5218 and LS5218 ΔatoC were unable to grow on LA whereas LS5218 ΔfadE and LS5218 ΔatoC ΔfadE grew robustly (equivalent to the positive control—E. coli M142 with pJMR32) on LA as the sole carbon source. See Table 9. These experiments demonstrated that the fadE deletion was necessary for growth, but the role of the atoC deletion remained unclear. We generated growth curves on LA for strains LS5218 ΔfadE, LS5218 ΔatoC ΔfadE and M142 and found that LS5218 ΔfadE has a significantly longer lag period compared with LS5218 ΔatoC ΔfadE and M142 (data not shown). This indicates that in E. coli LS5218, a fadE deletion and an atoC deletion are beneficial.

TABLE 9
LS5218 Growth on LA.

| LS5218 Strains^{a} | Growth on LA |
|---|---|
| Wild Type | −− |
| ΔfadE | ++ |
| ΔatoC | −− |
| ΔfadE ΔatoC | ++ |
| M142 | ++ |

^{a}All strains carrying pJMR32;
−−, no growth;
++, robust growth

FadE is an acyl-CoA dehydrogenase enzyme that catalyzes the formation of a trans-2-enoyl-CoA from an acyl-CoA compound. Because the LA catabolic pathway terminates at the formation of 3HV-CoA, the final steps to be completed by the E. coli β-oxidation pathway would only involve fadBA, so it remains unclear as to why a fadE deletion is beneficial for growth. We hypothesize that FadE may be active towards LA-CoA, adding a double bond at the 2 position of the γ-ketovaleryl-CoA species and sequestering the molecule from further degradation. FadE is an inner membrane protein that has not been purified for in vitro characterization. Díaz-Mejía, J. J., Babu, M. & Emili, A. Computational and experimental approaches to chart the Escherichia coli cell-envelope-associated proteome and interactome. FEMS Microbiol. Rev. 33, 66-97 (2009).

The deletion of atoC was not a necessary mutation, but did confer a growth benefit. This mutation was isolated during through the directed evolution process because we were screening for mutants with reduced lag phases, thereby enriching our mutant population with strains containing the early termination sequence. Constitutive activation of the ato regulon by the atoC(Con) mutation in LS5218 causes an overexpression of an acetoacetyl-CoA transferase (encoded by atoDA), an acetyl-CoA acetyltransferase (encoded by atoB) and a short chain fatty acid transporter (encoded by atoE). We propose that the 3-ketovaleryl-CoA intermediate was diverted from the final cleavage step into central metabolites by AtoDA, releasing 3-ketovalerate. The sequestering of LA as 3-ketovalerate reduces overall carbon flow to central metabolites, stunting growth of the E. coli strains until they can adapt for the utilization of 3-ketovalerate. Reducing expression of AtoDA through the deletion of atoC would prevent the formation of the secondary pathway, allowing direct flux of LA to central metabolites. Additionally, AtoE is a short chain fatty acid transporter and overexpression could be causing an increase in the intracellular concentration of LA above a threshold LS5218 is capable of tolerating, causing an extended lag phase. Monitoring intracellular metabolites during the extended lag phase could be useful in isolating the exact cause when compared with the ΔatoC strains.

### Directed Evolution of E. coli LS5218

E. coli was grown at 37° C., unless otherwise stated. Sub-culturing experiments were done with a volume of 5 ml in glass test tubes (20×150 mm, Fisher Scientific) with 250 rpm agitation in a 126 shaker (New Brunswick Scientific, Edison, NJ). Starting media contained 20 mM LA and 40 mM acetate or 40 mM acetate only for negative control. Cultures were grown for 72 hours and optical density (OD) measurements taken with a Spectronic 20 (Milton Roy Company, Warminster, PA), then culture were diluted 1:100 into fresh media. Once the OD in the LA and acetate cultures exceeded the OD of the acetate only cultures, further growth media was 20 mM LA only. These cultures were incubated until turbidity was observed visually, then diluted 1:100 into fresh media. This occurred for a total of 14 dilutions steps in LA media, spanning two weeks.

Plasmids were prepped (QIAprep® Miniprep Kits, Qiagen) and sequenced (Functional Biosciences) to find mutations. Plasmids were cured out of mutate strains through serial culturing in rich media (LB broth) and patch plated on LB and LBₖₐₙ₅₀.

### Developing a Growth-Coupled Strain

To further increase the yield of 2-butanone from LA, three strategies were computationally evaluated for producing 3-ketovalerate (3KV) from 3-ketovaleryl-CoA (3KV-CoA). The first method uses a thioesterase to hydrolyze 3KV-CoA to 3KV, which results in a strain incapable of growth without another carbon source but is theoretically capable of complete conversion of LA to butanone. The other two strategies couple butanone production to energy generation and/or cell growth. For example, the CoA moiety from 3KV-CoA can be transferred to succinate, thereby generating 3KV, via a succinyl-CoA transferase encoded by PcaIJ in Pseudomonas putida. With the addition of an equimolar feed of succinate and deletion of all reactions forming succinyl-CoA (SUCOAS, AKGDH, 3OXCOAT, PPCSCT) other than the PcaIJ reaction, it was determined that the maximum growth rate (0.48 hr⁻¹) required production of butanone at a rate of 0.25 mmol gDW⁻¹ hr⁻¹ (data not shown). While promising, this approach required a large number of deletions and maximum growth occurred with a relatively low amount of butanone production. Alternatively, it was found that butanone production could be coupled to acetate assimilation via E. coli's native acetyl-CoA transferase, AtoDA. By knocking out acetate fermentation (reactions ACKr and PTAr) and acetyl-CoA synthesis from acetate (reaction ACS), the only way for E. coli to make acetyl-CoA was through the transfer of CoA from 3HV-CoA to exogenous acetate. In this case, a maximum predicted 2-butanone production rate of 10 mmol gDW⁻¹ hr⁻¹ was achieved (i.e. complete bioconversion) with a maximum predicted growth rate of 0.21 hr⁻¹. This approach predicted the possibility of a strongly growth-coupled bioconversion of LA to butanone. This growth-coupled strain, eMEK8, was constructed by deleting ackApta and acs in strain eMEK4.

In a separate evolution experiment using E. coli K12 MG1655, a variant of the pJMR032 plasmid was discovered with a single nucleotide mutation causing a V111F variant on the Rep101 protein that improved catabolism of LA. The V111F mutation was the re-introduced into the Rep101 gene into pJMR032 creating plasmid pJMR032QC. This mutation significantly increased the growth rate of eMEK8 when compared to eMEK8 containing the plasmid without the V11F mutation (data not shown). Because a mutation in the origin is likely to affect the copy number of the plasmid, qPCR determined that the mutant plasmid had a copy number threefold higher than the original plasmid (data not shown).

To experimentally test the growth coupling strategy, cultures of eMEK8 with plasmids pJMR032QC and pJMR095 were grown in minimal media using LA and/or acetate at the carbon source. These data (not shown) revealed no growth of eMEK8 on LA or acetate alone but significant growth when both substrates are supplied, indicating that growth of eMEK8 is successfully coupled to LA metabolism. The effect of different ratios of LA and acetate on the production of 2-butanone was then examined. Importantly, in the case where LA was supplied at half the molar ratio as acetate (1:0.5), the LA was consumed in its entirety while acetate remained in the media. When supplied in equimolar ratios, the acetate and LA consumption are nearly identical (˜15 mM) and the measured yield of 2-butanone on LA is 76% (855 mg/L or 11.9 mM). The results from these experiments with the growth-coupled strain stand in stark contrast to the data from the non-growth-coupled strain, where all the supplied acetate was consumed in every case.

What is claimed is:

1. A genetically modified host cell transformed to contain and express a heterologous recombinant expression vector comprising at least one first promoter operably linked to at least three nucleotide sequences selected from the group consisting of a nucleotide sequence having at least 80% sequence identity to SEQ. ID. NO: 1 (lvaA), a nucleotide sequence having at least 80% sequence identity to SEQ. ID. NO: 3 (lvaB), a nucleotide sequence having at least 80% sequence identity to SEQ. ID. NO: 5 (lvaC), a nucleotide sequence having at least 80% sequence identity to SEQ. ID. NO: 7 (lvaD), and a nucleotide sequence having at least 80% sequence identity to SEQ. ID. NO: 9 (lvaE);
wherein the host cell is negative for at least one gene selected from the group consisting of fadA, fadE, fadI, fadR atoB, and atoC.

2. The genetically modified host cell of claim 1, wherein the host cell is fadE negative.

3. The genetically modified host cell of claim 1, wherein the host cell is atoC negative.

4. The genetically modified host cell of claim 1, wherein the host cell is fadE and atoC negative.

5. The genetically modified host cell of claim 1, wherein the host cell is fadA, fadI or atoB negative.

6. The genetically modified host cell of claim 1, wherein the host cell is fadA, fadI and atoB negative.

7. The genetically modified host cell of claim 1, wherein the host cell is fadR negative.

8. The genetically modified host cell of claim 1, wherein the host cell comprises an increased copy number of nucleotide sequences encoding FadB and/or FadJ as compared to the wild-type of the host cell.

9. The genetically modified host cell of claim 1, wherein the host cell is selected from a group consisting of an archaeal cell, a bacterial cell, and a eukaryotic cell.

10. The genetically modified host cell of claim 1, wherein the cell is selected from a group consisting of a bacterial cell and a eukaryotic single-cell organism.

11. The genetically modified host cell of claim 1, wherein the host cell constitutively expresses acetoacetyl-CoA transferase.

12. The genetically modified host cell of claim 1, wherein the heterologous recombinant expression vector comprises at least four nucleotide sequences selected from the group consisting of lvaA, lvaB, lvaC, lvaD, and lvaE.

13. The genetically modified host cell of claim 1, wherein the heterologous recombinant expression vector comprises five nucleotide sequences selected from the group consisting of lvaA, lvaB, lvaC, lvaD, and lvaE.

14. A method of catabolizing levulinic acid, the method comprising culturing a genetically modified host cell transformed to contain and express a heterologous recombinant expression vector comprising at least one promoter operably linked to at least three nucleotide sequences selected from the group consisting of a nucleotide sequence having at least 80% sequence identity to SEQ. ID. NO: 1 (lvaA), a nucleotide sequence having at least 80% sequence identity to SEQ. ID. NO: 3 (lvaB), a nucleotide sequence having at least 80% sequence identity to SEQ. ID. NO: 5 (lvaC), a nucleotide sequence having at least 80% sequence identity to SEQ. ID. NO: 7 (lvaD), and a nucleotide sequence having at least 80% sequence identity to SEQ. ID. NO: 9 (lvaE), in a medium containing levulinic acid, under conditions and for a time wherein at least a portion of the levulinic acid is catabolized by action of the genetically modified host cell.

15. A method of inducing a host cell to make 2-butanone from levulinic acid, the method comprising:
introducing into the host cell a heterologous operon encoding genes whose encoded proteins enable the host cell to catabolize levulinic acid into 3-hydroxyvaleryl-CoA (3HV-CoA);
upregulating expression of 3-hydroxyacyl-CoA dehydrogenase in the host cell to drive oxidation of at least a portion of 3HV-CoA to 3-ketovaleryl-CoA (3KV-CoA);
wherein the host cell expresses a nucleotide sequence encoding acetoacetyl-CoA transferase (atoDA) to drive conversion of at least a portion of the 3KV-CoA into 3-ketovalerate; and
wherein the host cell expresses a nucleotide sequence encoding acetoacetate decarboxylase (adc) to drive conversion of at least a portion of the 2-ketovalerate into butanone.

16. The method of claim 15, wherein the host cell is acetyl-CoA synthetase (Acs)-negative, phosphotransacetylase (Pta)-negative, and/or acetate kinase (Ack)-negative.

17. A combination of recombinant expression vectors, the combination comprising one or more expression vectors, each vector having one or more promoters operably linked to one or more nucleotide sequences selected from the group consisting of a nucleotide sequence having at least 80% sequence identity to SEQ. ID. NO: 1 (lvaA), a nucleotide sequence having at least 80% sequence identity to SEQ. ID. NO: 3 (lvaB), a nucleotide sequence having at least 80% sequence identity to SEQ. ID. NO: 5 (lvaC), a nucleotide sequence having at least 80% sequence identity to SEQ. ID. NO: 7 (lvaD), and a nucleotide sequence having at least 80% sequence identity to SEQ. ID. NO: 9 (lvaE).

18. The combination of recombinant expression vectors of claim 17, in which at least one of the vectors, or additional vectors, comprises a nucleotide sequence encoding an acetoacetyl-CoA transferase, a short-chain thioesterase, and/or a succinyl-CoA transferase, operably linked to a promoter.

19. The combination of recombinant expression vectors of claim 17, in which at least one of the vectors, or an additional vector, comprises a nucleotide sequence encoding an acetoacetate decarboxylase and which is operably linked to a promoter.

20. The combination of recombinant expression vectors of claim 17, in which at least one of the vectors, or one or more additional vectors, comprises a nucleotide sequence encoding an acetoacetyl-CoA transferase and a nucleotide sequence encoding an acetoacetate decarboxylase, both of which nucleotide sequences are operably linked to one or more corresponding promoters.

21. The combination of recombinant expression vectors of claim 17, in which at least one of the vectors, or one or more additional vectors, comprises a nucleotide sequence encoding FadB and/or FadJ, both of which nucleotide sequences are operably linked to one or more corresponding promoters.
